(19) United States
(12) Patent Application Publication
(10) Pub. No.: US 2025/0273294 A1
(43) Pub. Date: Aug. 28, 2025

(54) METHODS AND SYSTEMS FOR DETECTION OF REVERSION MUTATIONS FROM GENOMIC PROFILING DATA

(71) Applicant: Foundation Medicine, Inc., Boston, MA (US)

(72) Inventors: Chenming CUI, Columbia, MD (US); James THORNTON, Tucson, AZ (US); Meijuan LI, Clarksville, MD (US); Shannon BAILEY, Burlington, MA (US); Yuting HE, Stamford, CT (US)

(73) Assignee: Foundation Medicine, Inc., Boston, MA (US)

(21) Appl. No.: 18/682,373

(22) PCT Filed: Aug. 8, 2022
(86) PCT No.: PCT/US2022/074669

§ 371 Date: Feb. 8, 2024

Related U.S. Application Data
(60) Provisional application No. 63/231,470, filed on Aug. 10, 2021.

(51) Int. Cl. G16B 20/20; C12Q 1/6855; G16H 20/10

(52) U.S. Cl. CPC G16B 20/20; C12Q 1/6855; G16H 20/10

(57) ABSTRACT

Methods for detection and classification of reversion mutations are described. The methods may comprise, for example, receiving sequence data for nucleic acid sequences that reside within one or more gene loci within a subgenomic interval in a sample from a subject; identifying a gene locus of the one or more gene loci for which the gene locus comprises two or more variant sequences; categorizing the two or more variant sequences in the gene locus according to a structural feature or functional effect; comparing the structural features or functional effects of the two or more categorized variant sequences in the gene locus; and classifying the two or more categorized variant sequences in the gene locus based on the comparison, where the classification indicates whether the two or more categorized variant sequences comprise a reversion mutation.

### CROSS-REFERENCE TO RELATED APPLICATIONS

This application is a U.S. national stage application under 35 U.S.C. § 371 of International Application No. PCT/US2022/074669, filed internationally on Aug. 8, 2022, which claims the priority benefit of U.S. Provisional Patent Application Ser. No. 63/231,470, filed Aug. 10, 2021, the contents of each of which are incorporated herein by reference in their entirety.

### FIELD OF THE INVENTION

The present disclosure relates generally to methods and systems for analyzing genomic profiling data, and more specifically to methods and systems for detecting reversion mutations using genomic profiling data.

### BACKGROUND

Patients with certain types of cancer, for example, a breast or ovarian cancer harboring mutations in tumor suppressor genes such as BRCA1 and/or BRCA2, often benefit from treatment with inhibitors of poly (ADP-ribose) polymerase (PARPi) or platinum compounds. However, secondary mutations in BRCA1 and/or BRCA2 can restore the expression of their associated homologous repair capabilities and allow tumors to avoid the beneficial effects of treatment with PARPi or platinum-based therapies. These secondary mutations are often referred to as reversion mutations. Existing methods for detecting reversion mutations (e.g., manual review and curation of variant sequence data) are complex, laborious, and inaccurate. An improved process for more accurate detection and classification of reversion mutations would be advantageous in enabling healthcare providers to differentiate between those patients that will likely respond successfully to a given treatment (e.g., PARPi or platinum-based therapies in the case of BRCA1 and/or BRCA2 mutations) and those who will likely not to such a treatment, and would thus enable more informed treatment decisions and improved treatment outcomes.

### BRIEF SUMMARY OF THE INVENTION

Disclosed herein are methods and systems for automated detection and classification of reversion mutations. The process utilizes an extensive variant sequence database derived from comprehensive genomic profiling studies to: (i) identify gene loci comprising at least two variant sequences, (ii) parse and compare the coding sequence (CDS) structural features (e.g., “insertion”, “substitution”, “deletion”, etc.) and translational or protein functional effects (e.g., “missense”, “nonsense”, etc.) of the two variants (including computational analysis of the cis/trans position of the two variants in some cases), and (iii) classify the type of reversion mutation. The disclosed methods and systems provide more accurate detection, classification, and reporting of reversion mutations (e.g., for a variety of tumor suppressor genes) that can impact patient treatment decisions and outcomes.

Disclosed herein are methods comprising: providing a plurality of nucleic acid molecules obtained from a sample from a subject; ligating one or more adapters onto one or more nucleic acid molecules from the plurality of nucleic acid molecules; amplifying nucleic acid molecules from the plurality of nucleic acid molecules; capturing nucleic acid molecules from the amplified nucleic acid molecules; sequencing, using a sequencer, the captured nucleic acid molecules to obtain a plurality of sequence reads that represent the captured nucleic acid molecules, wherein one or more of the plurality of sequencing reads overlap one or more gene loci within a subgenomic interval in the sample; receiving, at one or more processors, sequence read data for the plurality of sequence reads; identifying, using the one or more processors, a gene locus of the one or more gene loci for which the gene locus comprises two or more variant sequences; categorizing, using the one or more processors, the two or more variant sequences in the gene locus according to one or more attributes of the two or more variant sequences; comparing, using the one or more processors, the one or more attributes of the two or more categorized variant sequences in the gene locus; classifying, using the one or more processors, the two or more categorized variant sequences in the gene locus based on the comparison, wherein the classification indicates whether the two or more categorized variant sequences comprise a reversion mutation; and identifying the reversion mutation in a genomic profile associated with the subject.

In some embodiments, the subject is suspected of having or is determined to have cancer. In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs).

In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adapter sequences, substrate adapter sequences, or sample index sequences. In some embodiments, the captured nucleic acid molecules are capture from the amplified nucleic acid molecules by hybridization to one or more bait molecules. In some embodiments, the one or more bait molecules comprise nucleic acid molecules, and wherein each bait molecule comprises a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises use of a massively parallel sequencing (MPS) technique, and the massively parallel sequencing technique comprises next generation sequencing (NGS). In some embodiments, the sequencer comprises a massively parallel sequencer. In some embodiments, the one or more attributes of the two or more variant sequences comprise a structural feature or functional effect. In some embodiments, a structural feature of a variant sequence comprises a substitution mutation (point mutation), an intragenic deletion, an insertion/deletion (indel) mutation, a splice site alteration, or a copy number alteration. In some embodiments, a functional effect comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event. In some embodiments, the reversion mutation is classified, based on the comparison of the structural features or functional effects of the two or more categorized variant sequences in the gene locus, as: (i) a missense mutation that restores a deleterious mutation in the same codon, (ii) a non-frameshift deletion spanning a nucleic acid sequence region comprising a deleterious mutation in a gene locus, (iii) a second frameshift indel that restores the open reading frame that was disrupted by a first frameshift indel, (iv) a splice-site alteration occurring at a same exon as a deleterious mutation, (v) a copy number loss event occurring at a same exon as a deleterious mutation (vi) a synonymous mutation that occurs at a same amino acid position as a deleterious mutation, or (vii) an intragenic deletion event spanning a nucleic acid sequence region comprising a deleterious mutation. In some embodiments, the method further comprises generating, by the one or more processors, a report indicating whether or not a reversion mutation in present in a gene locus of the one or more gene loci. In some embodiments, the method further comprises transmitting the report to a healthcare provider. In some embodiments, the report is transmitted via a computer network or a peer-to-peer connection.

Disclosed herein are computer-implemented methods comprising: receiving, at one or more processors, sequence read data for a plurality of sequence reads that overlap one or more gene loci within a subgenomic interval in a sample from a subject; identifying, using the one or more processors, a gene locus of the one or more gene loci for which the gene locus comprises two or more variant sequences; categorizing, using the one or more processors, the two or more variant sequences in the gene locus according to one or more attributes of the two or more variant sequences; comparing, using the one or more processors, the one or more attributes of the two or more categorized variant sequences in the gene locus; and classifying, using the one or more processors, the two or more categorized variant sequences in the gene locus based on the comparison, wherein the classification indicates whether the two or more categorized variant sequences comprise a reversion mutation.

In some embodiments, the computer-implemented method further comprising identifying the reversion mutation in a genomic profile associated with the subject. In some embodiments, the one or more attributes of the two or more variant sequences comprise a structural feature or functional effect. In some embodiments, the one or more gene loci comprise a tumor suppressor gene or fragment thereof, a homologous recombination repair gene or fragment thereof, or any combination thereof. In some embodiments, the one or more gene loci comprise APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof. In some embodiments, a structural feature of a variant sequence comprises a substitution mutation (point mutation), an intragenic deletion, an insertion/deletion (indel) mutation, a splice site alteration, or a copy number alteration. In some embodiments, a functional effect of a variant sequence comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event. In some embodiments, the functional effect of a variant sequence comprises a frameshift mutation, wherein the frameshift mutation results from an insertion of one or more nucleotides, and wherein a total number of inserted nucleotides is not a multiple of 3. In some embodiments, the functional effect of a variant sequence comprises a frameshift mutation, wherein the frameshift mutation results from a deletion of one or more nucleotides, and wherein a total number of deleted nucleotides is not a multiple of 3.

In some embodiments, if a first variant sequence of the two or more variant sequences comprises a truncating event and a second variant sequence of the two or more variant sequences comprises a mutation that restores an open reading frame, a comparison of the structural features and functional effects of the truncating event and the open reading frame event is executed, and the reversion mutation is classified, based on the comparison, as: (i) a possible missense mutation that restores a deleterious mutation in the same codon, (ii) a possible non-frameshift deletion spanning a nucleic acid sequence region comprising a deleterious mutation in a gene locus, or (iii) a possible second frameshift indel that restores the open reading frame that was disrupted by a first frameshift indel. In some embodiments, if the truncating event comprises a nonsense substitution mutation, the mutation that restores the open reading frame comprises a missense substitution mutation, and a starting base position and an ending base position for the nonsense substitution mutation are located at a same corresponding amino acid position as the missense substitution mutation, the reversion mutation is classified as a missense mutation that restores a deleterious mutation in the same codon. In some embodiments, if the truncating event comprises a frameshift insertion mutation, the mutation that restores the open reading frame comprises a missense substitution mutation, and a starting base position and an ending base position for the missense substitution mutation are located at a same corresponding amino acid position as the frameshift insertion mutation, the reversion mutation is classified as a missense mutation that restores a deleterious mutation in the same codon. In some embodiments, if the truncating event comprises a frameshift deletion mutation, the mutation that restores the open reading frame comprises a missense substitution mutation, and a starting base position and an ending base position for the missense substitution mutation are located at a same corresponding amino acid position as the frameshift deletion mutation, the reversion mutation is classified as a missense mutation that restores a deleterious mutation in the same codon. In some embodiments, if the mutation that restores the open reading frame comprises a non-frameshift deletion mutation, and a starting base position and an ending base position for the truncating event are located between a starting base position and an ending base position of the non-frameshift deletion mutation, the reversion mutation is classified as a non-frameshift deletion spanning a nucleic acid sequence region comprising a deleterious mutation in the gene locus. In some embodiments, if the mutation that restores an open reading frame comprises a non-frameshift deletion mutation, and the truncating mutation comprises an insertion mutation having a starting base position and an ending base position that are between a starting base position and an ending base position for the non-frameshift deletion mutation, the reversion mutation is classified as a non-frameshift deletion spanning a nucleic acid sequence region comprising a deleterious mutation in the gene locus.

In some embodiments, if both a first variant sequence and a second variant sequence of the two or more categorized variant sequences comprise a truncating event, a comparison of the structural features or functional effects of the first truncating event and the second truncating event is executed, and the reversion mutation is classified, based on the comparison, as a possible second frameshift indel that restores an open reading frame that was disrupted by a first frameshift indel. In some embodiments, if both the first truncating event and the second truncating event are indel mutations, the indel mutation corresponding to the second truncating event occurs at a position upstream from a stop codon for the indel mutation corresponding to the first truncating event, and if a net change in sequence length caused by the first and second truncating events is divisible by 3, the reversion mutation is classified as a second frameshift indel that restores an open reading frame that was disrupted by a first frameshift indel.

In some embodiments, if a first variant sequence of the two or more variant sequences comprises a deleterious truncating event and a second variant sequence of the two or more variant sequences comprises a splice site alteration, a comparison of the structural features or functional effects of the deleterious truncating event and the splice site alteration is executed, and the reversion mutation is classified, based on the comparison, as a splice site alteration affecting the same exon as the deleterious truncation event.

In some embodiments, if a first variant sequence of the two or more variant sequences comprises a deleterious truncating event and a second variant sequence of the two or more variant sequences comprises an intragenic deletion, and a starting base position and ending base position of the truncating event are between a starting base position and ending base position of the intragenic deletion, a comparison of the structural features or functional effects of the deleterious truncating event and the intragenic deletion is executed, and the reversion mutation is classified, based on the comparison, as the intragenic deletion spanning a nucleic acid sequence region comprising the deleterious truncating event.

In some embodiments, if a first variant sequence of the two or more variant sequences comprises a truncating event and a second variant sequence of the two or more variant sequences comprises a copy number loss event, a comparison of the structural features or functional effects of the truncating event and the copy number loss event is executed, and the reversion mutation is classified, based on the comparison, as a possible copy number loss of a complete exon comprising the truncating event. In some embodiments, if the truncating event has a starting base position and an ending base position located between a starting base position and an ending base position for the copy number loss event, and the copy number loss event comprises loss of a complete exon, the reversion mutation is classified as a copy number loss of a complete exon comprising the truncating event.

In some embodiments, if a first variant sequence of the two or more variant sequences comprises a truncating event and a second variant sequence of the two or more variant sequences comprises a non-truncating event, a comparison of the structural features or functional effects of the truncating event and the non-truncating event is executed, and the reversion mutation is classified, based on the comparison, as a possible synonymous mutation occurring at the same amino acid position as a deleterious mutation. In some embodiments, if the truncating event comprises a synonymous or nonsense mutation and the non-truncating event comprises a missense mutation, and wherein if the truncating and non-truncating events occur within a same codon, the reversion mutation is classified as a synonymous mutation occurring at the same amino acid position as a deleterious mutation. In some embodiments, if the truncating event comprises a frameshift insertion mutation and the non-truncating event comprises a synonymous mutation, and wherein if the non-truncating synonymous mutation comprises an insertion that occurs within a same nucleotide range as the truncating frameshift insertion, the reversion mutation is classified as a synonymous mutation occurring at the same amino acid position as a deleterious mutation. In some embodiments, if the truncating event comprises a frameshift deletion mutation and the non-truncating event comprises a synonymous mutation, and wherein if the non-truncating synonymous mutation comprises an insertion that occurs within a same nucleotide range as the truncating frameshift deletion, the reversion mutation is classified as a synonymous mutation occurring at the same amino acid position as a deleterious mutation.

In some embodiments, if a first variant sequence of the two or more variant sequences comprises a truncating event and a second variant sequence of the two or more variant sequences comprises a splice site alteration, a comparison of the structural features or functional effects of the truncating event and the splice site alteration is executed, and the reversion mutation is classified, based on the comparison, as a possible intragenic deletion spanning a nucleic acid sequence region comprising a deleterious mutation. In some embodiments, if the splice site alteration comprises an intragenic deletion that results in in-frame exon skipping of the exon in which the truncating event occurs, the reversion mutation is classified as an intragenic deletion spanning a nucleic acid sequence region comprising a deleterious mutation.

In some embodiments, the computer-implemented method further comprises selecting a cancer treatment for the subject based on the classification of the reversion mutation. In some embodiments, the cancer treatment comprises a treatment for a cancer caused by mutations in one or more genes of a homologous recombination repair (HRR) pathway. In some embodiments, the cancer treatment comprises a treatment for ovarian carcinoma, prostate carcinoma, or breast carcinoma. In some embodiments, the cancer treatment comprises use of a poly (ADP-ribose) polymerase inhibitor (PARPi), a platinum compound, a targeted immunotherapy treatment, or any combination thereof.

In some embodiments, the detection and classification of the reversion mutation is performed without any manual curation of the sequence read data. In some embodiments, the computer-implemented method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the computer-implemented method further comprises extracting nucleic acid molecules from the sample. In some embodiments, the nucleic acid molecules comprise tumor nucleic acid molecules. In some embodiments, the nucleic acid molecules comprise non-tumor nucleic acid molecules. In some embodiments, nucleic acid molecules comprise deoxyribonucleic acid (DNA) molecules. In some embodiments, the DNA molecules comprise cell-free DNA (cfDNA) molecules. In some embodiments, the DNA molecules comprise circulating tumor DNA (ctDNA). In some embodiments, the nucleic acid molecules comprise ribonucleic acid (RNA) molecules. In some embodiments, the computer-implemented method further comprises ligating one or more adapters to the nucleic acid molecules. In some embodiments, the computer-implemented method further comprises amplifying the nucleic acid molecules. In some embodiments, the computer-implemented method further comprises capturing a subset of nucleic acid molecules. In some embodiments, the captured subset of nucleic acid molecules are captured by hybridization to one or more bait molecules. In some embodiments, the computer-implemented method further comprises sequencing the captured nucleic acid molecules extracted from the sample to provide the sequence read data. In some embodiments, the sequencing is performed using a massively parallel sequencer. In some embodiments, the sequencing comprises targeted sequencing. In some embodiments, the targeted sequencing comprises targeted sequencing of one or more genes associated with cancer, or portions thereof. In some embodiments, the targeted sequencing comprises targeted sequencing of one or more exon regions. In some embodiments, the computer-implemented method further comprises generating, by the one or more processors, a report that comprises a list of reversion mutations detected in the sample. In some embodiments, the computer-implemented method further comprises transmitting the report to a healthcare provider. In some embodiments, the report is transmitted via a computer network or a peer-to-peer connection.

In some embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test. In some embodiments, the computer-implemented method further comprises selecting an anti-cancer agent, administering an anti-cancer agent, or applying an anti-cancer treatment to the subject based on the genomic profile

Disclosed herein are methods of selecting a treatment for a cancer in a subject, comprising: responsive to detecting and classifying a reversion mutation in a sample from the subject, selecting a cancer treatment for the subject, wherein the reversion mutation is identified and classified according to any of the methods described herein. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the cancer treatment comprises a treatment for a cancer caused by mutations in one or more genes of a homologous recombination repair (HRR) pathway. In some embodiments, the cancer treatment comprises a treatment for ovarian carcinoma, prostate carcinoma, or breast carcinoma. In some embodiments, the cancer treatment comprises use of a poly (ADP-ribose) polymerase inhibitor (PARPi), a platinum compound, a targeted immunotherapy treatment, or any combination thereof.

Also disclosed herein are methods of treating a cancer in a subject, comprising: responsive to detecting and classifying a reversion mutation in a sample from the subject, administering an effective amount of a cancer therapy to the subject, wherein the reversion mutation is identified and classified according to any of the method described herein. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the cancer treatment comprises a treatment for a cancer caused by mutations in one or more genes of a homologous recombination repair (HRR) pathway. In some embodiments, the cancer treatment comprises a treatment for ovarian carcinoma, prostate carcinoma, or breast carcinoma. In some embodiments, the cancer treatment comprises use of a poly (ADP-ribose) polymerase inhibitor (PARPi), a platinum compound, a targeted immunotherapy treatment, or any combination thereof.

Disclosed herein are systems comprising: one or more processors; and a memory communicatively coupled to the one or more processors and configured to store instructions that, when executed by the one or more processors, cause the system to: receive sequence read data for a plurality of sequence reads that overlap one or more gene loci within a subgenomic interval in a sample from a subject; identify a gene locus of the one or more gene loci for which the gene locus comprises two or more variant sequences; categorize the two or more variant sequences in the gene locus according to one or more attributes of the two or more variant sequences; compare the one or more attributes of the two or more categorized variant sequences in the gene locus; and classify the two or more categorized variant sequences, based on the comparison, wherein the classification indicates whether the two or more categorized variant sequences comprise a reversion mutation.

In some embodiments, the one or more attributes of the two or more variant sequences comprise a structural feature of functional effect. In some embodiments, the system further comprises a sequencer. In some embodiments, the sequencer comprises a massively parallel sequencer. In some embodiments, the massively parallel sequencer comprises a next generation sequencer. In some embodiments, the one or more gene loci comprise a tumor suppressor gene or fragment thereof, a homologous recombination repair gene or fragment thereof, or any combination thereof. In some embodiments, the one or more gene loci comprise APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof. In some embodiments, a structural feature of a variant sequence comprises a substitution mutation (point mutation), an intragenic deletion, an insertion/deletion (indel) mutation, a splice site alteration, or a copy number alteration. In some embodiments, a functional effect of a variant sequence comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event.

In some embodiments, if a first variant sequence of the two or more variant sequences comprises a truncating event and a second variant sequence of the two or more variant sequences comprises a mutation that restores an open reading frame, a comparison of the structural features and functional effects of the truncating event and the open reading frame event is executed, and the reversion mutation is classified, based on the comparison, as: (i) a possible missense mutation that restores a deleterious mutation in the same codon, (ii) a possible non-frameshift deletion spanning a nucleic acid sequence region comprising a deleterious mutation in a gene locus, or (iii) a possible second frameshift indel that restores the open reading frame that was disrupted by a first frameshift indel. In some embodiments, if the truncating event comprises a nonsense substitution mutation, the mutation that restores the open reading frame comprises a missense substitution mutation, and a starting base position and an ending base position for the nonsense substitution mutation are located at a same corresponding amino acid position as the missense substitution mutation, the reversion mutation is classified as a missense mutation that restores a deleterious mutation in the same codon. In some embodiments, if the truncating event comprises a frameshift insertion mutation, the mutation that restores the open reading frame comprises a missense substitution mutation, and a starting base position and an ending base position for the missense substitution mutation are located at a same corresponding amino acid position as the frameshift insertion mutation, the reversion mutation is classified as a missense mutation that restores a deleterious mutation in the same codon. In some embodiments, if the truncating event comprises a frameshift deletion mutation, the mutation that restores the open reading frame comprises a missense substitution mutation, and a starting base position and an ending base position for the missense substitution mutation are located at a same corresponding amino acid position as the frameshift deletion mutation, the reversion mutation is classified as a missense mutation that restores a deleterious mutation in the same codon. In some embodiments, if the mutation that restores the open reading frame comprises a non-frameshift deletion mutation, and a starting base position and an ending base position for the truncating event are located between a starting base position and an ending base position of the non-frameshift deletion mutation, the reversion mutation is classified as a non-frameshift deletion spanning a nucleic acid sequence region comprising a deleterious mutation in the gene locus. In some embodiments, if the mutation that restores an open reading frame comprises a non-frameshift deletion mutation, and the truncating mutation comprises an insertion mutation having a starting base position and an ending base position that are between a starting base position and an ending base position for the non-frameshift deletion mutation, the reversion mutation is classified as a non-frameshift deletion spanning a nucleic acid sequence region comprising a deleterious mutation in the gene locus.

In some embodiment, if both a first variant sequence and a second variant sequence of the two or more categorized variant sequences comprise a truncating event, a comparison of the structural features or functional effects of the first truncating event and the second truncating event is executed, and the reversion mutation is classified, based on the comparison, as a possible second frameshift indel that restores an open reading frame that was disrupted by a first frameshift indel. In some embodiments, if both the first truncating event and the second truncating event are indel mutations, the indel mutation corresponding to the second truncating event occurs at a position upstream from a stop codon for the indel mutation corresponding to the first truncating event, and if a net change in sequence length caused by the first and second truncating events is divisible by 3, the reversion mutation is classified as a second frameshift indel that restores an open reading frame that was disrupted by a first frameshift indel.

In some embodiments, if a first variant sequence of the two or more variant sequences comprises a deleterious truncating event and a second variant sequence of the two or more variant sequences comprises a splice site alteration, a comparison of the structural features or functional effects of the deleterious truncating event and the splice site alteration is executed, and the reversion mutation is classified, based on the comparison, as a splice site alteration affecting the same exon as the deleterious truncation event.

In some embodiments, if the first variant sequence of the two or more variant sequences comprises a deleterious truncating event and a second variant sequence of the two or more variant sequences comprises an intragenic deletion, and a starting base position and ending base position of the truncating event is between a starting base position and ending base position of the intragenic deletion, a comparison of the structural features or functional effects of the deleterious truncating event and the intragenic deletion is executed, and the reversion mutation is classified, based on the comparison, as the intragenic deletion spanning a nucleic acid sequence region comprising the deleterious truncating event.

In some embodiments, if a first variant sequence of the two or more variant sequences comprises a truncating event and a second variant sequence of the two or more variant sequences comprises a copy number loss event, a comparison of the structural features or functional effects of the truncating event and the copy number loss event is executed, and the reversion mutation is classified, based on the comparison, as a possible copy number loss of a complete exon comprising the truncating event. In some embodiments, if the truncating event has a starting base position and an ending base position located between a starting base position and an ending base position for the copy number loss event, and the copy number loss event comprises loss of a complete exon, the reversion mutation is classified as a copy number loss of a complete exon comprising the truncating event.

In some embodiments, if a first variant sequence of the two or more variant sequences comprises a truncating event and a second variant sequence of the two or more variant sequences comprises a non-truncating event, a comparison of the structural features or functional effects of the truncating event and the non-truncating event is executed, and the reversion mutation is classified, based on the comparison, as a possible synonymous mutation occurring at the same amino acid position as a deleterious mutation. In some embodiments, if the truncating event comprises a synonymous or nonsense mutation and the non-truncating event comprises a missense mutation, and wherein if the truncating and non-truncating events occur within a same codon, the reversion mutation is classified as a synonymous mutation occurring at the same amino acid position as a deleterious mutation. In some embodiments, if the truncating event comprises a frameshift insertion mutation and the non-truncating event comprises a synonymous mutation, and wherein if the non-truncating synonymous mutation comprises an insertion that occurs within a same nucleotide range as the truncating frameshift insertion, the reversion mutation is classified as a synonymous mutation occurring at the same amino acid position as a deleterious mutation. In some embodiments, if the truncating event comprises a frameshift deletion mutation and the non-truncating event comprises a synonymous mutation, and wherein if the non-truncating synonymous mutation comprises an insertion that occurs within a same nucleotide range as the truncating frameshift deletion, the reversion mutation is classified as a synonymous mutation occurring at the same amino acid position as a deleterious mutation.

In some embodiments, if a first variant sequence of the two or more variant sequences comprises a truncating event and a second variant sequence of the two or more variant sequences comprises a splice site alteration, a comparison of the structural features or functional effects of the truncating event and the splice site alteration is executed, and the reversion mutation is classified, based on the comparison, as a possible intragenic deletion spanning a nucleic acid sequence region comprising a deleterious mutation. In some embodiments, if the splice site alteration comprises an intragenic deletion that results in in-frame exon skipping of the exon in which the truncating event occurs, the reversion mutation is classified as an intragenic deletion spanning a nucleic acid sequence region comprising a deleterious mutation.

Also disclosed herein are non-transitory computer-readable storage media storing one or more programs, the one or more programs comprising instructions, which when executed by one or more processors of a computer system, cause the computer system to: receive sequence read data for a plurality of sequence reads that overlap one or more gene loci within a subgenomic interval in a sample from a subject; identify a gene locus of the one or more gene loci for which the gene locus comprises two or more variant sequences; categorize the two or more variant sequences in the gene locus according to one or more attributes of the two or more variant sequences; compare the one or more attributes of the two or more categorized variant sequences in the gene locus; and classify the two or more categorized variant sequences, based on the comparison, wherein the classification indicates whether the two or more categorized variant sequences comprise a reversion mutation.

In some embodiment, the one or more attributes of the two or more variant sequences comprise a structural feature or functional effect. In some embodiments, the one or more gene loci comprise a tumor suppressor gene or fragment thereof, a homologous recombination repair gene or fragment thereof, or any combination thereof. In some embodiments, the one or more gene loci comprise APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof. In some embodiments, a structural feature of a variant sequence comprises a substitution mutation (point mutation), an intragenic deletion, an insertion/deletion (indel) mutation, a splice site alteration, or a copy number alteration. In some embodiments, a functional effect of a variant sequence comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event.

In some embodiments, if a first variant sequence of the two or more variant sequences comprises a truncating event and a second variant sequence of the two or more variant sequences comprises a mutation that restores an open reading frame, a comparison of the structural features and functional effects of the truncating event and the open reading frame event is executed, and the reversion mutation is classified, based on the comparison, as: (i) a possible missense mutation that restores a deleterious mutation in the same codon, (ii) a possible non-frameshift deletion spanning a nucleic acid sequence region comprising a deleterious mutation in a gene locus, or (iii) a possible second frameshift indel that restores the open reading frame that was disrupted by a first frameshift indel. In some embodiments, if the truncating event comprises a nonsense substitution mutation, the mutation that restores the open reading frame comprises a missense substitution mutation, and a starting base position and an ending base position for the nonsense substitution mutation are located at a same corresponding amino acid position as the missense substitution mutation, the reversion mutation is classified as a missense mutation that restores a deleterious mutation in the same codon. In some embodiments, if the truncating event comprises a frameshift insertion mutation, the mutation that restores the open reading frame comprises a missense substitution mutation, and a starting base position and an ending base position for the missense substitution mutation are located at a same corresponding amino acid position as the frameshift insertion mutation, the reversion mutation is classified as a missense mutation that restores a deleterious mutation in the same codon. In some embodiments, if the truncating event comprises a frameshift deletion mutation, the mutation that restores the open reading frame comprises a missense substitution mutation, and a starting base position and an ending base position for the missense substitution mutation are located at a same corresponding amino acid position as the frameshift deletion mutation, the reversion mutation is classified as a missense mutation that restores a deleterious mutation in the same codon. In some embodiments, if the mutation that restores the open reading frame comprises a non-frameshift deletion mutation, and a starting base position and an ending base position for the truncating event are located between a starting base position and an ending base position of the non-frameshift deletion mutation, the reversion mutation is classified as a non-frameshift deletion spanning a nucleic acid sequence region comprising a deleterious mutation in the gene locus. In some embodiments, if the mutation that restores an open reading frame comprises a non-frameshift deletion mutation, and the truncating mutation comprises an insertion mutation having a starting base position and an ending base position that are between a starting base position and an ending base position for the non-frameshift deletion mutation, the reversion mutation is classified as a non-frameshift deletion spanning a nucleic acid sequence region comprising a deleterious mutation in the gene locus.

In some embodiments, if both a first variant sequence and a second variant sequence of the two or more categorized variant sequences comprise a truncating event, a comparison of the structural features or functional effects of the first truncating event and the second truncating event is executed, and the reversion mutation is classified, based on the comparison, as a possible second frameshift indel that restores an open reading frame that was disrupted by a first frameshift indel. In some embodiments, if both the first truncating event and the second truncating event are indel mutations, the indel mutation corresponding to the second truncating event occurs at a position upstream from a stop codon for the indel mutation corresponding to the first truncating event, and if a net change in sequence length caused by the first and second truncating events is divisible by 3, the reversion mutation is classified as a second frameshift indel that restores an open reading frame that was disrupted by a first frameshift indel.

In some embodiments, if a first variant sequence of the two or more variant sequences comprises a deleterious truncating event and a second variant sequence of the two or more variant sequences comprises a splice site alteration, a comparison of the structural features or functional effects of the deleterious truncating event and the splice site alteration is executed, and the reversion mutation is classified, based on the comparison, as a splice site alteration affecting the same exon as the deleterious truncation event.

In some embodiments, if a first variant sequence of the two or more variant sequences comprises a deleterious truncating event and a second variant sequence of the two or more variant sequences comprises an intragenic deletion, and the starting base position and ending base position of the truncating event is between the starting base position and ending base position of the intragenic deletion, a comparison of the structural features or functional effects of the deleterious truncating event and the intragenic deletion is executed, and the reversion mutation is classified, based on the comparison, as the intragenic deletion spanning a nucleic acid sequence region comprising the deleterious truncating event.

In some embodiments, if a first variant sequence of the two or more variant sequences comprises a truncating event and a second variant sequence of the two or more variant sequences comprises a copy number loss event, a comparison of the structural features or functional effects of the truncating event and the copy number loss event is executed, and the reversion mutation is classified, based on the comparison, as a possible copy number loss of a complete exon comprising the truncating event. In some embodiments, if the truncating event has a starting base position and an ending base position located between a starting base position and an ending base position for the copy number loss event, and the copy number loss event comprises loss of a complete exon, the reversion mutation is classified as a copy number loss of a complete exon comprising the truncating event.

In some embodiments, if a first variant sequence of the two or more variant sequences comprises a truncating event and a second variant sequence of the two or more variant sequences comprises a non-truncating event, a comparison of the structural features or functional effects of the truncating event and the non-truncating event is executed, and the reversion mutation is classified, based on the comparison, as a possible synonymous mutation occurring at the same amino acid position as a deleterious mutation. In some embodiments, if the truncating event comprises a synonymous or nonsense mutation and the non-truncating event comprises a missense mutation, and wherein if the truncating and non-truncating events occur within a same codon, the reversion mutation is classified as a synonymous mutation occurring at the same amino acid position as a deleterious mutation. In some embodiments, if the truncating event comprises a frameshift insertion mutation and the non-truncating event comprises a synonymous mutation, and wherein if the non-truncating synonymous mutation comprises an insertion that occurs within a same nucleotide range as the truncating frameshift insertion, the reversion mutation is classified as a synonymous mutation occurring at the same amino acid position as a deleterious mutation. In some embodiments, if the truncating event comprises a frameshift deletion mutation and the non-truncating event comprises a synonymous mutation, and wherein if the non-truncating synonymous mutation comprises an insertion that occurs within a same nucleotide range as the truncating frameshift deletion, the reversion mutation is classified as a synonymous mutation occurring at the same amino acid position as a deleterious mutation.

In some embodiments, if a first variant sequence of the two or more variant sequences comprises a truncating event and a second variant sequence of the two or more variant sequences comprises a splice site alteration, a comparison of the structural features or functional effects of the truncating event and the splice site alteration is executed, and the reversion mutation is classified, based on the comparison, as a possible intragenic deletion spanning a nucleic acid sequence region comprising a deleterious mutation. In some embodiments, if the splice site alteration comprises an intragenic deletion that results in in-frame exon skipping of the exon in which the truncating event occurs, the reversion mutation is classified as an intragenic deletion spanning a nucleic acid sequence region comprising a deleterious mutation.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A provides a non-limiting example of a process flowchart for detection and classification of reversion mutations.

FIG. 1B provides a non-limiting example of a process flowchart for detection and classification of reversion mutations.

FIG. 2 provides a non-limiting example of a process flowchart for detection and classification of Type 1 reversion mutations.

FIG. 3 provides a non-limiting example of a process flowchart for detection and classification of Type 2 reversion mutations.

FIG. 4 provides a non-limiting example of a process flowchart for detection and classification of Type 3 reversion mutations.

FIG. 5 provides a non-limiting example of a process flowchart for detection and classification of Type 4 reversion mutations.

FIG. 6 provides a non-limiting example of a process flowchart for detection and classification of Type 5 reversion mutations.

FIG. 7 provides a non-limiting example of a process flowchart for detection and classification of Type 6 reversion mutations.

FIG. 8 provides a non-limiting example of a process flowchart for detection and classification of Type 7 reversion mutations.

FIG. 9 provides a non-limiting schematic illustration of an electronic device or computer system according to examples of the present disclosure.

FIG. 10 provides a non-limiting schematic illustration of a computer network according to examples of the present disclosure.

FIG. 11 provides a non-limiting example of a Type 1 reversion mutation as described herein and as viewed in a graphical user interface (GUI) for a genomics data visualization tool.

FIG. 12 provides a non-limiting example of a Type 2 reversion mutation as described herein and as viewed in a graphical user interface (GUI) for a genomics data visualization tool.

FIG. 13 provides a non-limiting example of a Type 3 reversion mutation as described herein and as viewed in a graphical user interface (GUI) for a genomics data visualization tool.

### DETAILED DESCRIPTION

Methods and systems for detection and classification of reversion mutations are described that leverage an extensive variant sequence database derived from comprehensive genomic profiling studies to: (i) identify gene loci comprising at least two variant sequences, (ii) parse and compare the coding sequence (CDS) structural features (e.g., “insertion”, “substitution”, “deletion”, etc.) and translational or protein functional effects (e.g., “missense”, “nonsense”, etc.) of the two variants (including computational analysis of the cis/trans position of the two variants), and (iii) classify the type of reversion mutation. Compared to currently available methods, e.g., manual curation, the methods described herein provide more accurate results for detection of reversion mutations and can identify complex reversion mutations (e.g., reversion mutations of more complex structure) in complex and less apparent data sets. They also provide a more simplistic approach to identifying reverse mutations than the complex mechanisms that underlie currently available methods. Advantageously, the disclosed methods may be performed to automatically identify reversion mutations without any manual curation of sequence read data.

In some instances, for example, computer-implemented methods are described that comprise the use of one or more processors to: receive sequence data for nucleic acid sequences that reside within one or more gene loci within a subgenomic interval in a sample from a subject; identify a gene locus of the one or more gene loci for which the gene locus comprises two or more variant sequences; categorize the two or more variant sequences in the gene locus according to a structural feature or functional effect; compare the structural features or functional effects of the two or more categorized variant sequences in the gene locus; and classify the two or more categorized variant sequences in the gene locus based on the comparison, wherein the classification indicates whether the two or more categorized variant sequences comprise a reversion mutation.

In some instances, the one or more gene loci comprise a tumor suppressor gene or fragment thereof, a homologous recombination repair gene or fragment thereof, or any combination thereof. In some instances, the one or more gene loci comprise APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof.

In some instances, a structural feature (e.g., a CDS effect) of a variant sequence comprises a substitution mutation (point mutation), an intragenic deletion, an insertion/deletion (indel) mutation, a splice site alteration, or a copy number alteration.

In some instances, a translational or protein functional effect of a variant sequence comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event.

Comparison of the structural features and/or functional effects of the two or more variant sequences in a given gene locus, e.g., through the implementation of a logical decision tree process, then allows for the detection and classification of reversion mutations according to type, as will be described in more detail below.

The disclosed methods and systems eliminate the need for manual review of sequencing data and provide for improved consistency in the detection and reporting of reversion mutations (for a variety of tumor suppressor genes) that can impact patient treatment decisions and outcomes. For example, BRCA reversion mutations that restore protein function constitute a key tumor resistance mechanism to platinum-based chemotherapies and treatment with PARP inhibitors in cancers that exhibit BRCA mutations (Lin, et al. (2019), “BRCA reversion mutations in circulating tumor DNA predict primary and acquired resistance to the PARP inhibitor rucaparib in high-grade ovarian carcinoma”, Cancer Discovery 9.2: 210-219). Cancers that exhibit BRCA mutations may comprise a defective homologous recombination repair (HRR) mechanism and have been shown to be responsive to platinum-based chemotherapies and PARP inhibitors (Lin, et al. (2019), ibid.; Farmer, et al. (2005), “Targeting the DNA repair defect in BRCA mutant cells as a therapeutic strategy” Nature 434.7035: 917-921). However, the presence of a reversion mutation in a BRCA mutation-related cancer, e.g., a nonsense mutation or frameshift event that results in expression of a truncated BRCA protein, can lead to acquired resistance to PARP inhibitors. For example, cancer patients who harbor a BRCA deleterious mutation but no reversion mutation in the BRCA gene have significantly longer progression-free survival (PFS) after rucaparib treatment than those patients for which BRCA reversion mutations were identified. Similar results have been found in platinum-resistant or platinum-refractory subgroups of patients exhibiting a BRCA mutation-related cancer.

The disclosed methods and systems provide a means for reversion mutation screening and surveillance at the molecular level for cancer patients that will assist clinicians in making treatment decisions, not only for BRCA mutation-related cancers but also more generally. For example, reversion mutations have been reported in multiple HRR pathway genes for various cancers. In addition to reversion mutations in BRCA1 and BRCA2, reversion mutations in the RAD51C, RAD51D and PALB2 genes from ovarian, prostate and breast carcinomas have all been shown to be involved in this common mechanism of acquired resistance to platinum-based chemotherapies and PARP inhibitors (Norquist, et al. (2011), “Secondary somatic mutations restoring BRCA1/2 predict chemotherapy resistance in hereditary ovarian carcinomas”, J. Clinical Oncology 29.22: 3008; Patch, et al. (2015), “Whole-genome characterization of chemoresistant ovarian cancer.” Nature 521.7553: 489-494; Barber, et al. (2013), “Secondary mutations in BRCA2 associated with clinical resistance to a PARP inhibitor”, Journal of Pathology 229.3: 422-429; Goodall, et al. (2017), “Circulating cell-free DNA to guide prostate cancer treatment with PARP inhibition”, Cancer Discovery 7.9: 1006-1017; Kondrashova, et al. (2017), “Secondary somatic mutations restoring RAD51C and RAD51D associated with acquired resistance to the PARP inhibitor rucaparib in high-grade ovarian carcinoma”, Cancer Discovery 7.9: 984-998; Quigley, et al. (2017), “Analysis of circulating cell-free DNA identifies multiclonal heterogeneity of BRCA2 reversion mutations associated with resistance to PARP inhibitors”, Cancer Discovery 7.9: 999-1005).

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the term “deleterious mutation” refers to a nonsense, missense, frameshift, non-frameshift, splice-site alteration, synonymous, copy number alteration, or large structural rearrangement mutation that leads to partial or complete loss of function for a protein corresponding to the gene product for the gene in which a variant sequence occurs.

As used herein, the term “reversion mutation” refers to the presence of a second mutation in a gene that counteracts the structural feature or functional effect of a first mutation in the same gene to restore the original phenotypic trait (e.g., protein function) associated with the gene.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The section headings used herein are for organizational purposes only and are not to be construed as limiting the subject matter described.

### Structure Features & Functional Effects of Variant Sequences

The disclosed methods evaluate the structural features and functional effects of variant sequences identified in sequencing data by a sequencing data analysis pipeline for calling variant sequences. Examples of structural features of variant sequences include, but are not limited to, substitution mutations (point mutations), intragenic deletions (e.g., small deletions within a gene that inactivate the gene and have the same effect as other null mutations of that gene), insertion/deletion (indel) mutations (e.g., an insertion or deletion of multiple bases), splice site alterations (e.g., mutations that insert, delete, or changes a number of nucleotides in the specific site at which splicing takes place during the processing of precursor messenger RNA into mature messenger RNA), and copy number alterations (e.g., mutations that can include deletions, insertions, and duplications of entire exons or genes).

Examples of functional effects (e.g., translational and/or protein functional effects) include, but are not limited to, missense mutations (e.g., point mutations in which a single nucleotide change results in a codon that codes for a different amino acid in the corresponding protein), nonsense mutations (e.g., point mutations that result in a premature stop codon and a truncated and usually nonfunctional protein product), null mutations (e.g., mutations that lead to a gene not being transcribed into RNA and/or translated into a functional protein product), deleterious mutations (e.g., mutations that lead to partial or complete loss of function for a corresponding protein product), synonymous mutations (e.g., mutations which are “silent” and do not alter the amino acid sequence of a corresponding protein product), non-synonymous mutations (e.g., mutations that alter the amino acid sequence of a corresponding protein product), truncating events (e.g., mutations that result in a truncated protein product), non-truncating events (e.g., mutations that do not impact the length of the protein product), frameshift mutations (e.g., mutations (also called framing error mutations) that are caused by insertion or deletion of a number of nucleotides that is not divisible by three, and thus result in an altered translation of the variant sequence into protein that is different from the native or wild-type protein), non-frameshift mutations (e.g., mutations that do not result in a reading frameshift), exon skipping events (e.g., mutations that cause skipping of exons during translation, thereby leading to truncated but potentially still functional protein), or copy number loss events (e.g., mutations that result in a change in copy number for a gene or portion thereof).

### Methods for Detecting Reversion Mutations

The disclosed methods and systems use variant sequence data derived from nucleic acid sequencing workflows as input to identify and classify reversion mutation pairs. The variant sequence data file (e.g., an XML file) contains genomic variant details as determined by a sequencing data analysis pipeline for calling variant sequences. In addition to the variant sequences themselves, examples of variant sequence data that may be incorporated into the data file include, but are not limited to, structural features or coding sequence (CDS) effects, translational and/or protein functional effects, copy number variations, allele frequency, etc.

The computer-implemented reversion mutation detection process starts with the parsing of the CDS effects of variants co-occurring in a given gene to determine if a potential reversion mutation is present. The process first identifies variants that are derived from the same gene by parsing the gene symbol information from each short variant. For the gene(s) comprising multiple variants, truncating and non-truncating variants may be identified by parsing additional variant sequence information (e.g., functional effect, protein effect) from the variant sequence data file. The process determines if a truncating event has occurred from a short variant's functional effect, e.g., a “frameshift”, “nonsense”, or, in special cases, a “non-frameshift” alteration. In the case of “frameshift” or “nonsense” mutations, the variant may be identified as a truncating event. In the case of “non-frameshift” alterations, the protein effect may be parsed to determine if a stop codon has been incurred, in which case the variant may again be identified as truncating event. For example, if the functional effect of a short variant from the BRCA1 gene is “nonsense”, it may be identified as a truncating event.

The process then may determine if a co-occurring alteration corrects or reverts the original deleterious mutation, and classifies detected reversion mutations according to type. Table 1 provides a summary of the different types of reversion mutations that may be identified using the methods disclosed herein. Specific, non-limiting examples of Type 1-Type 7 reversion mutations are described below in Example 1.

TABLE 1
Reversion mutation classification.

| Reversion | |
|---|---|
| Type | Biomarker Definition |
| Type 1 | A missense mutation that restores a deleterious mutation ^{[1]} |
| | in the same codon, for example: |
| | A missense mutation that corrects a missense or nonsense |
| | mutation |
| | A missense mutation that corrects a frameshift incurred by an |
| | insertion |
| | A missense mutation that corrects a frameshift incurred by a |
| | deletion |
| Type 2 | A non-frameshift deletion spanning the region of a deleterious |
| | mutation ^{[1]} in the same gene. |
| Type 3 | A second frameshift indel that restores an open reading frame |
| | disrupted by a first frameshift indel. |
| Type 4 | Splice-site alteration occurring at the same exon as a |
| | deleterious mutation. |
| Type 5 | Copy number loss of an exon with a deleterious mutation. |
| Type 6 | Synonymous alteration occurring at the same amino acid |
| | position as a deleterious mutation. |
| Type 7 | An intragenic deletion event spanning a nucleic acid sequence |
| | region comprising a deleterious mutation. |

[1] Deleterious mutations include nonsense, missense, frameshift, non-frameshift, splice-site alteration, synonymous, copy number alteration, and large structural rearrangement mutations.
[2] Cis read number may be calculated by counting the number of reads that harbor both variants in the corresponding sequence data file (e.g., a Binary Alignment Map (BAM) file, etc.). Total read number may be calculated by counting the total number of reads covering a locus. The cis read percentage may be calculated as (cis read number)/(total read number)*100.
[3] The cis detection power may be restricted by read length. For example, the average read length may be ˜150 base pairs.

If a missense mutation and another deleterious mutation (e.g. a “nonsense”, “missense”, or “frameshift” mutation, or one of the other deleterious mutations listed above) are identified in the same codon (and possibly in the same sequence read (e.g., in a “cis” configuration)), these two mutations may be identified and classified as a Type 1 reversion mutation pair. The process determines if the two variants are harbored in the same sequence read by analysis of the aligned sequence read data stored in a sequencing data file (e.g., a BAM file, etc.). In the case of missense mutations, for example, variant sequences are passed to an “iterator” (e.g., a programmable pointer that can be used to loop through a list) that goes through each sequence read and attempts to locate the variant of interest on the sequence read by matching the variant sequence to a sequence region from the sequence read, where the sequence region is determined by variant position information. If both of the two variants are identified in the same sequence read, they are determined to be in “cis”. The iterator may then increase the count for the number of “cis” reads detected (i.e., sequence reads comprising both variants) and the total number of sequence reads analyzed for the genetic region (there is no minimum “cis” read fraction or threshold applied by the classifier). Determining if the two variants are in the “cis” configuration with each other increases the confidence level that a potential reversion mutation has occurred between two co-occurring alterations.

Co-occurring alterations that are in a “trans” configuration (i.e., occurring on the opposite or complementary strands at a given genomic locus, and thus appearing in different sequence reads) may result from sub-clonal events which have no reversion implications. In some instances, by applying the cis read criterion for determination of Type 1 reversions additional support may be provided that a potential reversion alteration is occurring. In this case, the number of cis reads and the total number of reads may be calculated without applying a read percentage threshold. Note that if the missense mutation occurs at the same nucleotide position, the cis read count cannot be determined. In some instances, the process may report a missense substitution at two consecutive codons when an indel occurs between the two codons. In some instances, a reversion mutation may generate a deleterious mutation by chance, in which case it is still identified as a reversion mutation but not reported.

If a non-frameshift deletion is identified that spans the region of a deleterious mutation in the same gene, these two mutations may be identified as a Type 2 reversion mutation pair, i.e., a non-frameshift event that counteracts the effect of the deleterious mutation. Note that the process may be unable to check if the two mutations are in cis due to the deletion event that spans the original deleterious mutation. Furthermore, there may be no threshold applied for minimum allele frequency in identifying either Type 2 or Type 3 reversion mutations.

If a frameshift indel is identified that occurs upstream of a stop codon introduced by a first frameshift indel, and the net change in sequence length is a multiple of 3, then the process may identify and classify the mutations as a Type 3 reversion mutation, i.e., a second frameshift indel that restores an open reading frame that was disrupted by a first frameshift indel.

Similarly, a Type 4 reversion mutation comprises a splice-site alteration occurring at the same exon as a deleterious mutation and counteracting the effect of the deleterious mutation. A base substitution, insertion, or deletion occurring at the splice donor or splice acceptor site of the same exon as a deleterious mutation (e.g., a frameshift mutation or nonsense mutation) is expected to result in exon skipping of the exon harboring the deleterious mutation. The expected gene product needs to be in-frame with the splice site mutation and expected exon skipping. A Type 5 reversion mutation comprises a copy number loss event that eliminates an exon comprising a deleterious mutation. The expected gene product needs to be in-frame with the resulting copy number loss. A Type 6 reversion mutation comprises a synonymous alteration occurring at the same amino acid position as a deleterious mutation. A Type 7 reversion mutation comprises an intragenic deletion event spanning a nucleic acid sequence region comprising a deleterious mutation. Intragenic deletions affecting the splice donor or splice acceptor site are expected to result in exon skipping of the exon harboring the deleterious mutation. The expected gene product needs to be in frame with the intragenic deletion.

In some instances, the disclosed methods may be applied to the detection and analysis of reversion mutations in at least 1, at least 2, at least 3, at least 4, at least 5, at least 6, at least 7, at least 8, at least 9, at least 10, at least 20, at least 30, at least 40, or more than 40 gene loci.

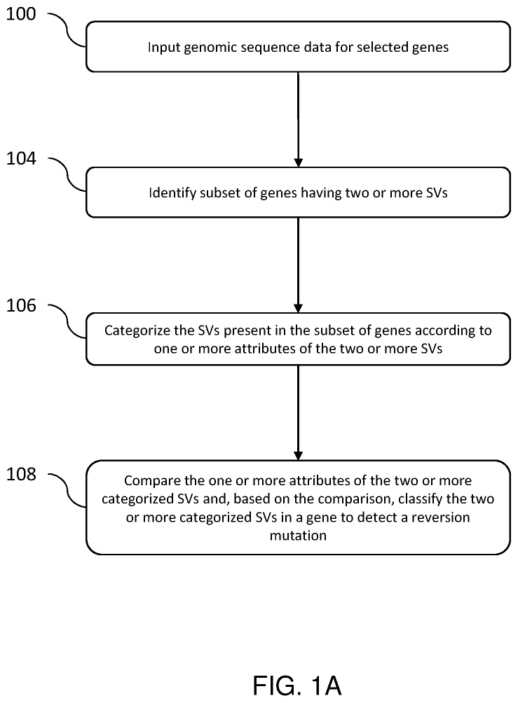

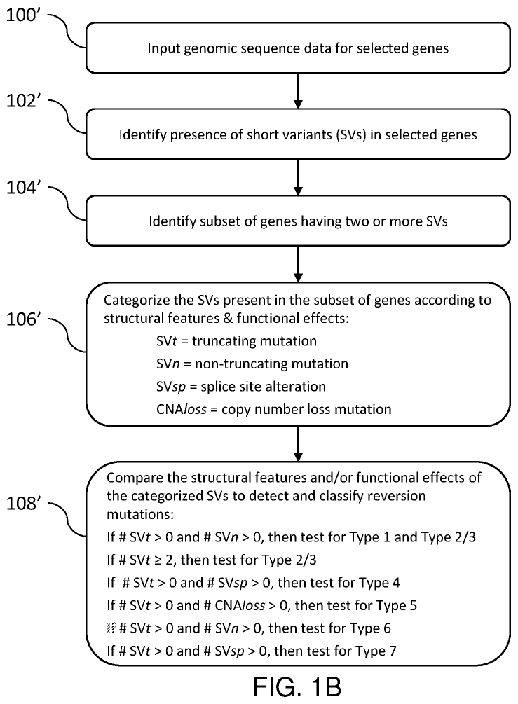

FIG. 1A provides a non-limiting example of a process flowchart for detection and classification of reversion mutations according to the present disclosure. The process begins at step 100 with the input of genomic sequence data generated by a nucleic acid sequencing workflow for one or more selected genes or gene loci. At step 102, the sequence data is analyzed to identify the presence of short variant sequences (SVs) in the one or more selected genes or gene loci using, e.g., any of a variety of variant sequence calling procedures known to those of skill in the art, and to identify a subset of the one or more selected genes or gene loci that comprise two or more variant sequences. At step 106, the variant sequences identified in genes or gene loci that comprise two or more variants may then be categorized according to one or more attributes (e.g., a structural and/or functional effect). At step 108, the one or more attributes of the two or more categorized variant sequences may be compared and, based on the comparison, the two or more SVs identified in a given gene or gene locus may be classified, where the classification indicates whether a reversion mutation is present and optionally provides a classification of the reversion mutation according to type.

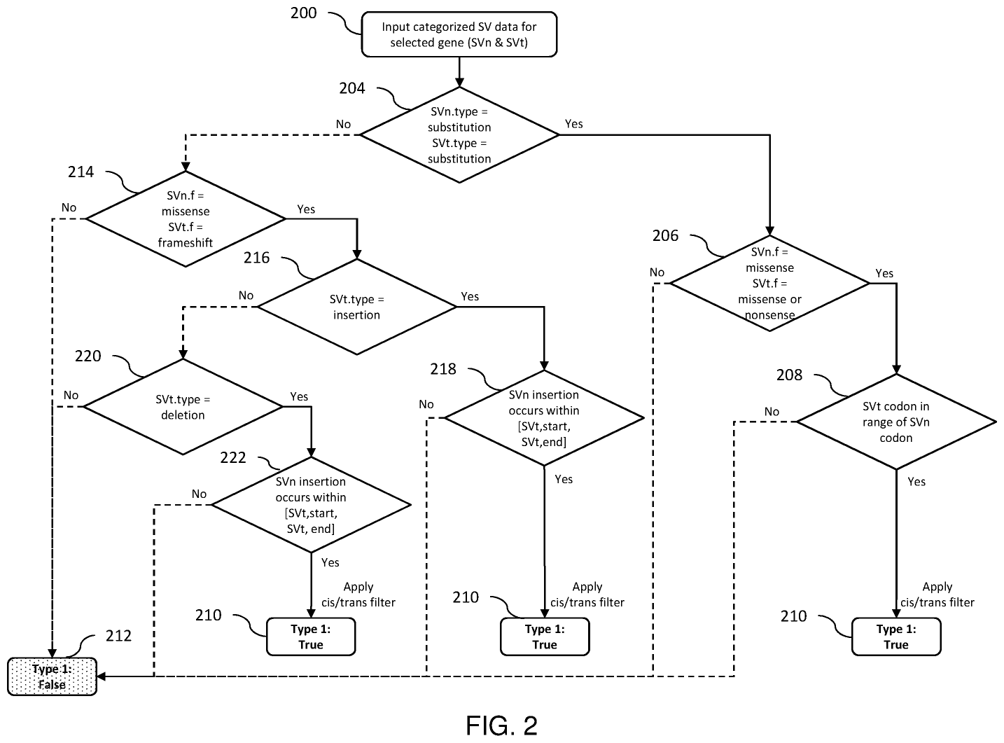

FIG. 1B provides another non-limiting example of a process flowchart for detection and classification of reversion mutations according to the present disclosure (e.g., where the one or more attributes used to categorize short variant sequences (SVs) comprise a structural feature and/or functional effect). The process begins at step 100′ with the input of genomic sequence data (e.g., in the form of a variant sequence XML file) generated by a nucleic acid sequencing workflow for one or more selected genes or gene loci. At step 102′, the sequence data is analyzed to identify the presence of short variant sequences (SVs) in the one or more selected genes or gene loci using, e.g., any of a variety of variant sequence calling procedures known to those of skill in the art. Examples include, but are not limited to, the variant calling procedures described in U.S. Pat. Nos. 9,340,830 and 9,792,403, and in International Patent Application Publication No. WO2021/086335. At step 104′, the subset of genes or gene loci that comprise two or more variant sequences are identified, e.g., by parsing the gene symbol information for each short variant that is included in the genomic sequence data file. At step 106′, the variant sequences identified in genes or gene loci that comprise two or more variants may then be categorized according to their respective functional effects, e.g., as truncating events (SVt), non-truncating events (SVn), splice site alterations (SVsp), or copy number loss events (CNAloss). At step 108′, a series of logical tests may be applied to compare the structural features and/or functional effects of the two variants identified in a given gene or gene locus and thereby detect the presence of a reversion mutation (and optionally, to classify the reversion according to type). The logical tests applied at step 108′ of FIG. 1B will be described in more detail in the process flowcharts illustrated in FIGS. 2-8.

FIG. 2 provides a non-limiting example of a process flowchart for detection and classification of Type 1 reversion mutations, i.e., missense mutations that counteract the effect of a deleterious mutation in the same codon, if the two or more categorized variants identified comprise at least one truncating event (SVt) and at least one non-truncating event (SVn). The process begins at step 200 with the input of the categorized SV data for a given gene or gene locus. At step 204, the structural features of the two variants (SVn.type and SVt.type) may be compared to determine if they both comprise base substitutions. If so, the functional effects of the two variants (SVn.f and SVt.f) may be compared at step 206 to determine if the non-truncating event comprises a missense mutation and the truncating event comprises a missense mutation or a nonsense mutation. If the answer is affirmative, then the position of the base substitution in each variant sequence may be compared at step 208 to determine if they occur within the same codon. Again, if the answer is affirmative, the cis/trans relationship of the two variants may be determined as described above. In some instances, if they reside in a cis configuration the pair of variants may be classified as a Type 1 reversion mutation at step 210.

If, at step 204 of FIG. 2, it is determined that the truncating event is not a base substitution, then the functional effects of the two variants (SVn.f and SVt.f) may be compared at step 214 to determine if the non-truncating event comprises a missense mutation and the truncating event comprises a frameshift mutation. If the truncating event is determined to be a frameshift mutation, the structural features of the frameshift variant (SVt.type) may be examined at step 216 to determine if the frameshift mutation is the result of one or more base insertions. If the answer is affirmative, then the position of the base insertion in the truncating event may be compared to the position of the base substitution in the non-truncating event at step 218 to determine if they occur within the same codon. Again, if the answer is affirmative, the cis/trans relationship of the two variants may be determined as described above. In some instances, if they reside in a cis configuration the pair of variants may be classified as a Type 1 reversion mutation at step 210.

If, at step 216 of FIG. 2, it is determined that the frameshift mutation is not the result of a base insertion, the structural features of the frameshift variant (SVt.type) may be examined at step 220 to determine if the frameshift mutation is the result of one or more base deletions. If the answer is affirmative, then the position of the base deletion in the truncating event may be compared to the position of the base substitution in the non-truncating event at step 222 to determine if they occur within the same codon. Again, if the answer is affirmative, the cis/trans relationship of the two variants may be determined as described above. In some instances, if they reside in a cis configuration the pair of co-occurring variants may be classified as a Type 1 reversion mutation at step 210. All of the remaining decision tree paths illustrated in FIG. 2 lead to a call of “Type 1=False” at step 212 for the two variants identified in the gene or gene locus being analyzed. If more than two variants are identified in a given gene or gene locus, all possible pairwise combinations of the more than two variants may be subjected to the same analysis to determine whether or not a Type 1 reversion has occurred.

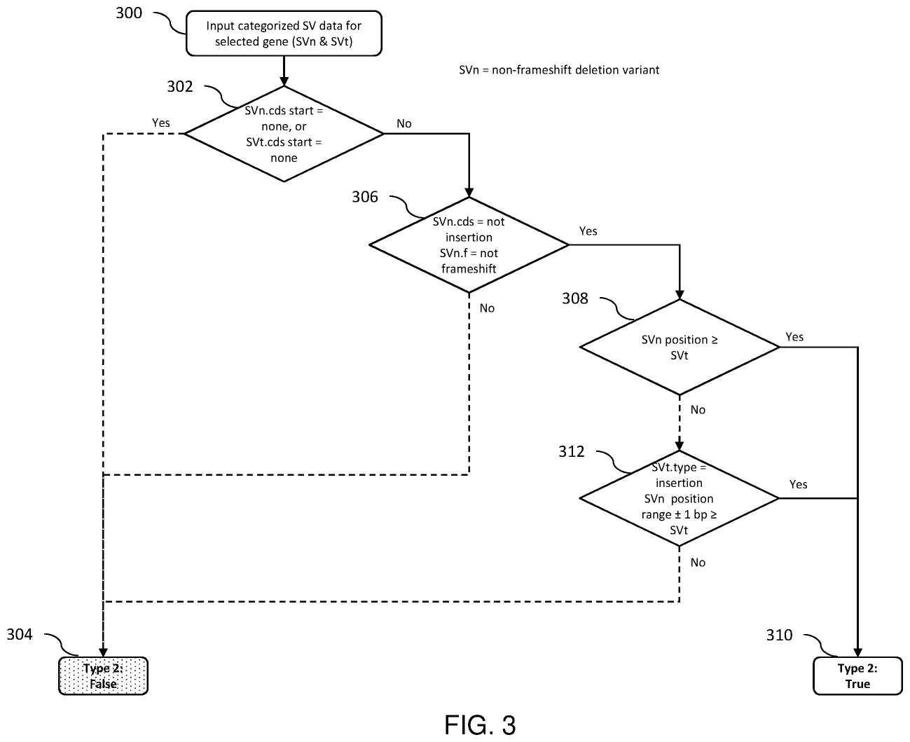

FIG. 3 provides a non-limiting example of a process flowchart for detection and classification of Type 2 reversion mutations, i.e., a non-frameshift deletion spanning the region of a deleterious mutation in the same gene, if the two or more categorized variants identified comprise at least one truncating event (SVt) and a non-truncating event (SVn) that comprises a non-frameshift deletion variant. The process begins at step 300 with the input of the categorized SV data for a given gene or gene locus. At step 302, the starting positions for the truncating and non-truncating variants in the CDS data may be examined to determine if the start positions were identified. If not, the variants may be classified as “Type 2=False” at step 304. If the start positions for the two variants have been identified, the structural features (CDS effect) and functional effects of the non-truncating alteration (SVn.cds and SVn.f, respectively) may be examined at step 306 to determine if the non-truncating variant is an insertion or causes a frameshift. If not, the position of the truncating variant may be compared to the starting and ending positions of the non-truncating variant at step 308. If the non-truncating variant sequence spans the truncating variant sequence, the two variants may be classified as a Type 2 reversion mutation at step 310. If not, the structural features of the truncating (SVt.type) and non-truncating variants (SVn) may be compared at step 312 to determine if the truncating variant is an insertion and is located within ±1 bp of the location range of the non-truncating variant. If so, the two co-occurring variants may be classified as a Type 2 reversion mutation at step 310. All of the remaining decision tree paths illustrated in FIG. 3 lead to a call of “Type 2=False” at step 304 for the two variants identified in the gene or gene locus being analyzed. If more than two variants are identified in a given gene or gene locus, all possible pairwise combinations of the more than two variants may be subjected to the same analysis to determine whether or not a Type 2 reversion has occurred.

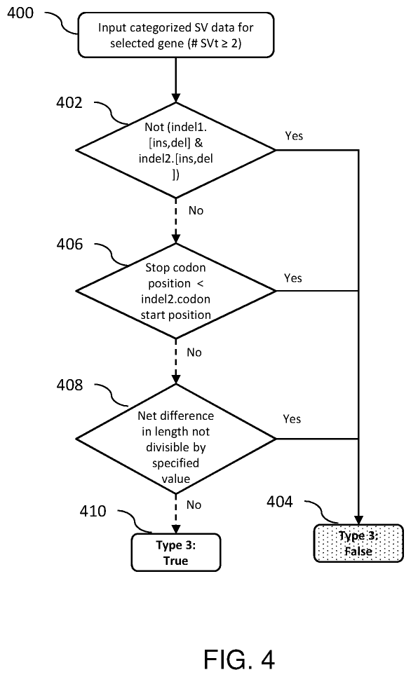

FIG. 4 provides a non-limiting example of a process flowchart for detection and classification of Type 3 reversion mutations, i.e., frameshift indels that restores an open reading frame disrupted by a first frameshift indel, if the two or more categorized variants identified comprise at least two truncating events (SVt). The process begins at step 400 with the input of the categorized SV data for a given gene or gene locus. At step 402, the structural features of the two variants may be compared to determine if they are both indel mutations (Indel1.[ins,del] and Indel2.[ins,del]). If not, a call of “Type 3=False” may be made at step 404. If so, the structural features of the two indel mutations may be compared at step 406 to determine if the second indel occurs at a position upstream from a stop codon for the first indel. If so, a call of “Type 3=False” may be made at step 404. If not, the structural features of the two indel mutations may be compared at step 408 to determine if the net difference in sequence length for the two variants is a multiple of a specified value (e.g., a value of 3). If not, a call of “Type 3=False” may be made at step 404. If so, the pair of co-occurring variants may be classified as a Type 3 reversion mutation at step 410. If more than two variants comprising truncating events are identified in a given gene or gene locus, all possible pairwise combinations of the more than two variants may be subjected to the same analysis to determine whether or not a Type 3 reversion has occurred.

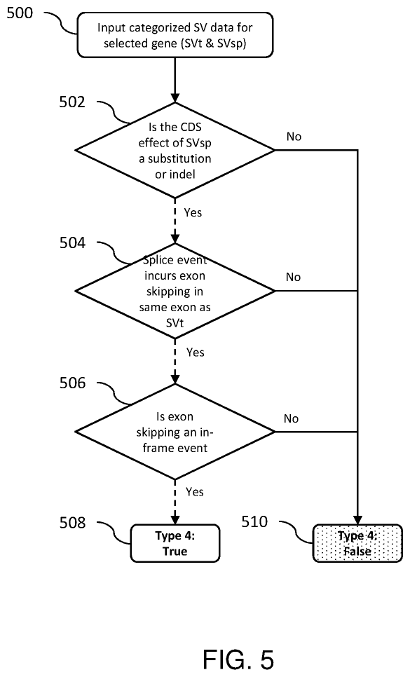

FIG. 5 provides a non-limiting example of a process flowchart for detection and classification of Type 4 reversion mutations, i.e., splice-site alterations occurring in the same exon as a deleterious mutation, if the two or more categorized variants identified comprise a truncating event (SVt) and a splice site alteration (SVsp). The process begins at step 500 with the input of the categorized SV data for a given gene or gene locus. At step 502, the structural features of the two variants may be compared to determine if the CDS effect of the SVsp alteration is a substitution or indel. If so, the structural features of the two variants may be compared at step 504 to determine if the splicing event results in exon skipping for the same exon that is impacted by the splice site alteration. If so, structural features and functional effects of the two variants may be compared at step 506 to determine if the exon skipping comprises an in-frame event. If the answer is affirmative, the pair of variants may be classified as a Type 4 reversion at step 508. All of the remaining decision tree paths illustrated in FIG. 5 lead to a call of “Type 4=False” at step 510 for the two co-occurring variants identified in the gene or gene locus being analyzed. If more than two variants are identified in a given gene or gene locus, all possible pairwise combinations of the more than two variants may be subjected to the same analysis to determine whether or not a Type 4 reversion has occurred.

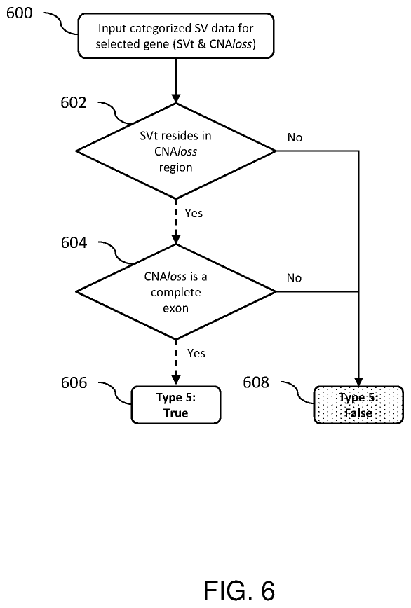

FIG. 6 provides a non-limiting example of a process flowchart for detection and classification of Type 5 reversion mutations, i.e., copy number loss of an exon comprising a deleterious mutation, if the two or more categorized variants identified comprise a truncating event (SVt) and a copy number loss event (CNAloss). The process begins at step 600 with the input of the categorized SV data for a given gene or gene locus. At step 602, the structural features of the two variants may be compared to determine if the truncating event resides in the same sequence region as the copy number loss event. If the answer is affirmative, the structural features of the copy number loss event may be examined at step 604 to determine if the sequence region comprising the copy number loss constitutes loss of a complete exon. If so, the pair of co-occurring variants may be classified as a Type 5 reversion mutation at step 606. All of the remaining decision tree paths illustrated in FIG. 6 lead to a call of “Type 5=False” at step 608 for the two co-occurring variants identified in the gene or gene locus being analyzed. If more than two variants are identified in a given gene or gene locus, all possible pairwise combinations of the more than two variants may be subjected to the same analysis to determine whether or not a Type 5 reversion has occurred.

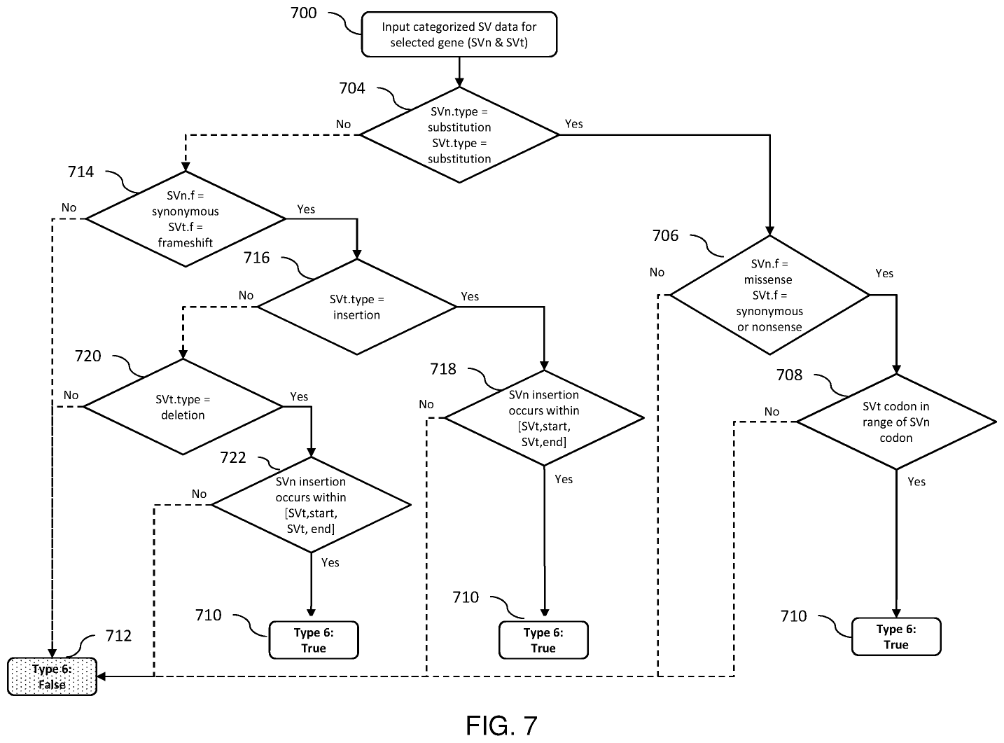

FIG. 7 provides a non-limiting example of a process flowchart for detection and classification of Type 6 reversion mutations, i.e., synonymous mutations that counteract the effect of a deleterious mutation in the same codon, if the two or more categorized variants identified comprise at least one truncating event (SVt) and at least one non-truncating event (SVn). The process begins at step 700 with the input of the categorized SV data for a given gene or gene locus. At step 704, the structural features of the two variants (SVn.type and SVt.type) may be compared to determine if they both comprise base substitutions. If so, the functional effects of the two variants (SVn.f and SVt.f) may be compared at step 706 to determine if the non-truncating event comprises a missense mutation and the truncating event comprises a synonymous mutation or a nonsense mutation. If the answer is affirmative, then the position of the base substitution in each variant sequence may be compared at step 708 to determine if they occur within the same codon. Again, if the answer is affirmative, the pair of variants may be classified as a Type 6 reversion mutation at step 710.

If, at step 704 of FIG. 7, it is determined that the truncating event is not a base substitution, then the functional effects of the two variants (SVn.f and SVt.f) may be compared at step 714 to determine if the non-truncating event comprises a synonymous mutation and the truncating event comprises a frameshift mutation. If the truncating event is determined to be a frameshift mutation, the structural features of the frameshift variant (SVt.type) may be examined at step 716 to determine if the frameshift mutation is the result of one or more base insertions. If the answer is affirmative, then the position of the base insertion in the truncating event may be compared to the position of the base substitution in the non-truncating event at step 718 to determine if they occur within the same codon. Again, if the answer is affirmative, the pair of variants may be classified as a Type 6 reversion mutation at step 710.

If, at step 716 of FIG. 7, it is determined that the frameshift mutation is not the result of a base insertion, the structural features of the frameshift variant (SVt.type) may be examined at step 720 to determine if the frameshift mutation is the result of one or more base deletions. If the answer is affirmative, then the position of the base deletion in the truncating event may be compared to the position of the base substitution in the non-truncating event at step 722 to determine if they occur within the same codon. Again, if the answer is affirmative, pair of co-occurring variants may be classified as a Type 6 reversion mutation at step 710. All of the remaining decision tree paths illustrated in FIG. 7 lead to a call of “Type 6=False” at step 712 for the two variants identified in the gene or gene locus being analyzed. If more than two variants are identified in a given gene or gene locus, all possible pairwise combinations of the more than two variants may be subjected to the same analysis to determine whether or not a Type 6 reversion has occurred.

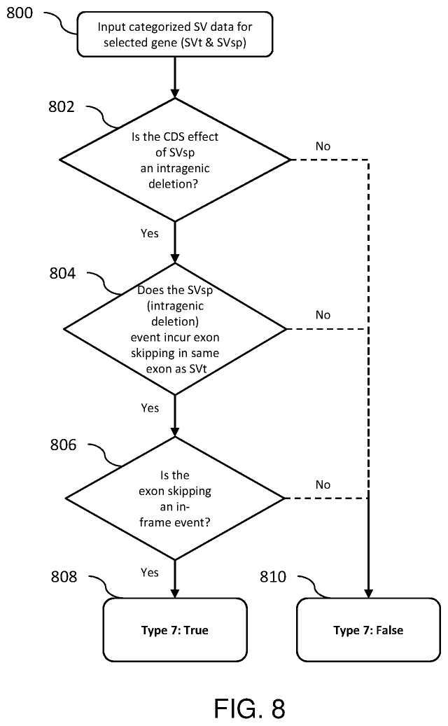

FIG. 8 provides a non-limiting example of a process flowchart for detection and classification of Type 7 reversion mutations, i.e., intragenic deletions that encompass a deleterious mutation. Intragenic deletions affecting the splice donor or splice acceptor site are expected to result in exon skipping of the exon harboring the deleterious mutation. The expected gene product needs to be in frame with respect to the intragenic deletion. The process begins at step 800 with the input of the categorized SV data for a given gene or gene locus. At step 802, the structural features of the two variants (SVt and SVsp) may be compared to determine if the CDS effect of the SVsp alteration is an intragenic deletion. If so, the structural features of the two variants may be compared at step 804 to determine if the splicing event (intragenic deletion) results in exon skipping for the same exon that is impacted by the SVt truncation event. If so, structural features and functional effects of the two variants is compared at step 806 to determine if the exon skipping comprises an in-frame event. If the answer is affirmative, the pair of variants may be classified as a Type 7 reversion at step 808. All of the remaining decision tree paths illustrated in FIG. 8 lead to a call of “Type 7=False” at step 810 for the two co-occurring variants identified in the gene or gene locus being analyzed. If more than two variants are identified in a given gene or gene locus, all possible pairwise combinations of the more than two variants may be subjected to the same analysis to determine whether or not a Type 7 reversion has occurred.

### METHODS OF USE

As noted above, the disclosed methods enable partially- or fully-automated detection and classification of reversion mutations based on variant sequence data identified by sequencing data analysis pipelines. The ability to detect and classify reversion mutations while reducing or eliminating the need for laborious and costly manual review of sequencing data can impact patient treatment decisions and outcomes by identifying patients that will likely not respond to conventional treatments for a given cancer.

In some instances, as noted above, the disclosed methods may be applied to the detection and analysis of reversion mutations in at least 1, at least 2, at least 3, at least 4, at least 5, at least 6, at least 7, at least 8, at least 9, at least 10, at least 20, at least 30, at least 40, at least 50, or more than 50 gene loci.

In some instances, as noted above, the gene loci used for the detection of reversion mutations comprise one or more of APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof.

In some instances, the disclosed methods may be used to identify reversion mutations in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify reversion mutations in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-10, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K6, PIGF, PTCH, RAF, RANKL, RET, ROS1, SLAMF7, VEGF, VEGFA, or VEGFB gene locus, or any combination thereof.

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for detection and/or classification of reversion mutations may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for detection and/or classification of reversion mutations may be used to select a subject (e.g., a patient) for a clinical trial based on the detection of a reversion mutation in one or more gene loci. In some instances, patient selection for clinical trials based on, e.g., identification of reversion mutations at one or more gene loci, may accelerate the development of targeted therapies and improve the healthcare outcomes for treatment decisions.

In some instances, the disclosed methods for detection and/or classification of reversion mutations may be used to select an appropriate treatment (e.g., an anti-cancer treatment or anti-cancer therapy (also referred to herein as a cancer treatment or cancer therapy)) for a subject. In some instances, for example, the anti-cancer treatment (or anti-cancer therapy) may comprise use of a poly (ADP-ribose) polymerase inhibitor (PARPi), a platinum compound, chemotherapy, radiation therapy, a targeted therapy (e.g., immunotherapy, surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfe (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for detection and/or classification of reversion mutations may be used in treating a disease (e.g., a cancer) in a subject. For example, in response to detecting a reversion mutation in one or more gene loci using any of the methods disclosed herein, an effective amount of an anti-cancer therapy or anti-cancer treatment may be administered to the subject. In some instances, the disclosed methods may be used for adjusting a treatment (e.g., an anti-cancer treatment or anti-cancer therapy) for a subject (e.g., by adjusting a treatment dose and/or selecting a different treatment). In some instances, the disclosed methods may be used for monitoring disease (e.g., cancer) or disease progression (e.g., cancer progression).

In some instances, the detection and/or classification of reversion mutations using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for detection and/or classification of reversion mutations may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for detection and/or classification of reversion mutations as part of a genomic profiling process can improve the validity of, e.g., disease detection calls and treatment decisions, made on the basis of the genomic profile by, for example, independently confirming the presence of a reversion mutation in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering an anti-cancer agent or applying an anti-cancer treatment to the subject based on the generated genomic profile. An anti-cancer agent or anti-cancer treatment can refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used to detect and classify reversion mutations in any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc. In some instances, the sample is acquired from a liquid biopsy, which can be from, e.g., whole blood, blood plasma, blood serum, urine, stool, sputum, saliva, or cerebrospinal fluid.

In some instances, the sample comprises one or more premalignant or malignant cells. Premalignant, as used herein, refers to a cell or tissue that is not yet malignant but is poised to become malignant. In certain instances, the sample is acquired from a solid tumor, a soft tissue tumor, or a metastatic lesion. In certain instances, the sample is acquired from a hematologic malignancy or pre-malignancy. In other instances, the sample comprises a tissue or cells from a surgical margin. In certain instances, the sample comprises tumor-infiltrating lymphocytes. In some instances, the sample comprises one or more non-malignant cells. In some instances, the sample is, or is part of, a primary tumor or a metastasis (e.g., a metastasis biopsy sample). In some instances, the sample is obtained from a site (e.g., a tumor site) with the highest percentage of tumor (e.g., tumor cells) as compared to adjacent sites (e.g., sites adjacent to the tumor). In some instances, the sample is obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease, e.g., a hyperproliferative disease or a non-cancer indication. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with a cancer therapy (or cancer treatment). In some instances, the subject is in need of being or is being monitored for relapse of cancer. In some instances, the subject is in need of being monitored for minimum residual disease (MRD). In some instances, the subject has been, or is being treated, for cancer. In some instances, the subject has not been treated with a cancer therapy (or cancer treatment).

In some instances, the subject (e.g., a patient) has been previously treated with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise acquiring a yield value for the nucleic acid extracted from the sample and comparing the acquired value to a reference value. For example, if the acquired value is less than the reference value, the nucleic acids may be amplified prior to proceeding with library construction. In some instances, the disclosed methods may further comprise acquiring a value for the size (or average size) of nucleic acid fragments in the sample, and comparing the acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, sample barcodes, and/or unique molecular identifier sequences), size-selected (e.g., by preparative gel electrophoresis), and/or amplified (e.g., using PCR). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library can contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single individual. In some instances, a library can comprise nucleic acid molecules derived from more than one subject (e.g., 2, 3, 4, 5, 6, 7, 8, 9, 10, 20, 30 or more subjects), e.g., two or more libraries from different subjects can be combined to form a library having nucleic acid molecules from more than one subject. In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., bait molecule, is a capture oligonucleotide. In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one embodiment, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent sequence may comprise forward and reverse complement sequences for the same target nucleic acid molecule sequence, whereby the target capture reagent sequence with reverse-complemented target-specific sequences also carries reverse complement universal tails. This can lead to RNA transcripts that comprise the same strand, i.e., not complementary to each other.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

The disclosed methods may comprise the use of one, two, three, four, five, or more than five pluralities of target capture reagents, where each plurality is designed to capture target sequences in a different target category. In some instances, any combination of two, three, four, five, or more than five pluralities of target capture reagents can be used, for example, a combination of first and second pluralities of target capture reagents; first and third pluralities of target capture reagents; first and fourth pluralities of target capture reagents; first and fifth pluralities of target capture reagents; second and third pluralities of target capture reagents; second and fourth pluralities of target capture reagents; second and fifth pluralities of target capture reagents; third and fourth pluralities of target capture reagents; third and fifth pluralities of target capture reagents; fourth and fifth pluralities of target capture reagents; first, second and third pluralities of target capture reagents; first, second and fourth pluralities of target capture reagents; first, second and fifth pluralities of target capture reagents; first, second, third, and fourth pluralities of target capture reagents; first, second, third, fourth and fifth pluralities of target capture reagents, and so on.

In some instances, each of the first, second, third, fourth, and fifth, etc., pluralities of target capture reagents has a unique recovery efficiency. In some instances, at least two or three pluralities of target capture reagents have recovery efficiency values that differ.

In some instances, the value for recovery efficiency is modified by one or more of: differential representation of different target capture reagents, differential overlap of target capture reagent subsets, differential target capture reagent parameters, mixing of different target capture reagents, and/or using different types of target capture reagents. For example, a variation in recovery efficiency (e.g., relative sequence coverage of each target capture reagent/target category) can be adjusted, e.g., within a plurality of target capture reagents and/or among different pluralities of target capture reagents, by altering one or more of:
(i) Differential representation of different target capture reagents—The target capture reagent design to capture a given target (e.g., a target nucleic acid molecule) can be included in more/fewer number of copies to enhance/reduce relative target sequencing depths;
(ii) Differential overlap of target capture reagent subsets—The target capture reagent design to capture a given target (e.g., a target nucleic acid molecule) can include a longer or shorter overlap between neighboring target capture reagents to enhance/reduce relative target sequencing depths;
(iii) Differential target capture reagent parameters—The target capture reagent design to capture a given target (e.g., a target nucleic acid molecule) can include sequence modifications/shorter length to reduce capture efficiency and lower the relative target sequencing depths;
(iv) Mixing of different target capture reagents—Target capture reagents that are designed to capture different target sets can be mixed at different molar ratios to enhance/reduce relative target sequencing depths;
(v) Using different types of oligonucleotide target capture reagents—In certain instances, the target capture reagent can include:
(a) one or more chemically (e.g., non-enzymatically) synthesized (e.g., individually synthesized) target capture reagents,
(b) one or more target capture reagents synthesized in an array,
(c) one or more enzymatically prepared, e.g., in vitro transcribed, target capture reagents;
(d) any combination of (a), (b) and/or (c),
(e) one or more DNA oligonucleotides (e.g., a naturally or non-naturally occurring DNA oligonucleotide),
(f) one or more RNA oligonucleotides (e.g., a naturally or non-naturally occurring RNA oligonucleotide),
(g) a combination of (e) and (f), or
(h) a combination of any of the above.

The different oligonucleotide combinations can be mixed at different ratios, e.g., a ratio chosen from 1:1, 1:2, 1:3, 1:4, 1:5, 1:10, 1:20, 1:50; 1:100, 1:1000, or the like. In one embodiment, the ratio of chemically-synthesized target capture reagent to array-generated target capture reagent is chosen from 1:5, 1:10, or 1:20.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

Target capture reagents comprising DNA or RNA oligonucleotides can include naturally- or non-naturally-occurring nucleotides. In some instances, the target capture reagents include one or more non-naturally-occurring nucleotides to, e.g., increase melting temperature. Exemplary non-naturally occurring oligonucleotides include modified DNA or RNA nucleotides. Exemplary modified nucleotides (e.g., modified RNA or DNA nucleotides) include, but are not limited to, locked nucleic acids (LNAs), wherein the ribose moiety of an LNA nucleotide is modified with an extra bridge connecting the 2′ oxygen and 4′ carbon; peptide nucleic acids (PNAs), e.g., a PNA composed of repeating N-(2-aminoethyl)-glycine units linked by peptide bonds; a DNA or RNA oligonucleotide modified to capture low GC regions; a bicyclic nucleic acid (BNA); a cross-linked oligonucleotide; a modified 5-methyl deoxycytidine; and 2,6-diaminopurine. Other modified DNA and RNA nucleotides are known in the art.

In some instances, a substantially uniform or homogeneous coverage of a target sequence (e.g., a target nucleic acid molecule) is obtained. For example, within each target capture reagent/target category, uniformity of coverage can be optimized by modifying target capture reagent parameters, for example, by one or more of:
(i) Increasing/decreasing target capture reagent representation or overlap can be used to enhance/reduce coverage of targets (e.g., target nucleic acid molecules), which are under/over-covered relative to other targets in the same category;
(ii) For low coverage, hard to capture target sequences (e.g., high GC content sequences), expand the region being targeted with the target capture reagents to cover, e.g., adjacent sequences (e.g., less GC-rich adjacent sequences);
(iii) Modifying a target capture reagent sequence can be used to reduce secondary structure of the target capture reagent and enhance its recovery efficiency;
(iv) Modifying a target capture reagent length can be used to equalize melting hybridization kinetics of different target capture reagents within the same category. Target capture reagent length can be modified directly (by producing target capture reagents with varying lengths) or indirectly (by producing target capture reagents of consistent length, and replacing the target capture reagent ends with arbitrary sequence);
(v) Modifying target capture reagents of different orientation for the same target region (i.e. forward and reverse strand) may have different binding efficiencies. The target capture reagent with either orientation providing optimal coverage for each target may be selected;
(vi) Modifying the amount of a binding entity, e.g., a capture tag (e.g. biotin), present on each target capture reagent may affect its binding efficiency. Increasing/decreasing the tag level of target capture reagents targeting a specific target may be used to enhance/reduce the relative target coverage;
(vii) Modifying the type of nucleotide used for different target capture reagents can be used to affect binding affinity to the target, and enhance/reduce the relative target coverage; or
(viii) Using modified oligonucleotide target capture reagents, e.g., having more stable base pairing can be used to equalize melting hybridization kinetics between areas of low or normal GC content relative to high GC content.

In some instances, the disclosed methods thus comprise providing a selected set of nucleic acid molecules (e.g., a library catch) captured from one or a plurality of nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library catch. The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to determine, e.g., gene allele sequences at a plurality of gene loci. “Next-generation sequencing” (or “NGS”) as used herein, refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise acquiring a library comprising a plurality of nucleic acid molecules (e.g., DNA or RNA molecules) derived from a sample from a subject. In certain instances, the methods further comprise contacting the library with target capture reagents (e.g., using solution-based hybridization or solid-phase hybridization of nucleic acid molecules to target capture probes and/or sequencing primers), thereby providing a targeted library or targeted subgroup thereof (e.g., a library catch) of subject intervals (e.g., subgenomic sequences or target sequences) that can be sequenced. In certain instances, the methods further comprise acquiring a sequence read (e.g., using a next-generation sequencing method) for one or more subject intervals (target sequences) that correspond to one or more targeted genomic loci (e.g., gene loci) that may comprise an alteration (e.g., substitutions at one or more nucleotide positions, or an indel that results in a change in allele length). In certain instances, the disclosed methods further comprise aligning a read for the one or more subject intervals using an alignment method (e.g., an alignment method described herein). In certain instances, the methods further comprise identifying a nucleotide (or assigning a nucleotide value) for a given nucleotide position in a read for the one or more subject intervals, e.g., using a mutation calling method described herein. In certain instances, the methods may comprise re-sequencing all or a portion of the library catch.

In certain instances, the disclosed methods comprise one, two, three, four, five, or more of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules to provide a selected set of normal and/or tumor nucleic acid molecules, wherein said plurality of target capture reagents hybridize with the normal and/or tumor nucleic acid molecules, thereby providing a library catch; (c) separating the selected subset of the nucleic acid molecules (e.g., a library catch) from the hybridization mixture, e.g., by contacting the hybridization mixture with a binding entity that allows for separation of the target capture reagent/nucleic acid molecule hybrids from the hybridization mixture, (d) acquiring a read (e.g., a sequence read) for one or more subject intervals (e.g., one or more target or subgenomic sequences) from said library catch that may comprise an alteration (e.g., a somatic alteration or variant allele) using, e.g., a next-generation sequencing method; (e) aligning said sequence read using an alignment method (e.g., an alignment method described herein); and/or (f) assigning a nucleotide value for a nucleotide position of the subject interval (e.g., calling a mutation (e.g., using a Bayesian method or other method described herein)) from the sequence read.

In some instances, acquiring a read for the one or more subject intervals comprises sequencing a subject interval for at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., gene loci. In some instances, acquiring a read for the one or more subject intervals may comprise sequencing a subject interval for any number of loci, e.g., gene loci, within the range described in this paragraph, e.g., at least 2,850 gene loci.

In some instances, acquiring a read for the one or more subject intervals comprises sequencing a subject interval with a sequencing method that provides a read length (or average read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a read for the one or more subject intervals may comprise sequencing a subject interval with a sequencing method that provides a read length (or average read length) of any number of bases within the range described in this paragraph, e.g., a read length (or average read length) of 56 bases.

In some instances, acquiring a read for the one or more subject intervals comprises sequencing with at least 100× or more average depth. In some instances, acquiring a read for the subject interval comprises sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more average depth. In some instances, acquiring a read for the subject interval may comprise sequencing with an average depth having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, sequence reads from each of X unique subject intervals are aligned using from 1 to X unique alignment method(s), and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, or at least 1000. In some instances, subject intervals from at least X genomic loci (e.g., at least X microsatellite loci) are aligned using from 1 to X unique alignment method(s), and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, or at least 1000.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

In some instances, the disclosed methods may comprise the use of an alignment selector. An “alignment selector”, as used herein, refers to a parameter that allows or directs the selection of an alignment method, e.g., an alignment algorithm or parameter, that can optimize the sequencing performance for a subject interval. An alignment selector can be specific to, or selected as, a function of, e.g., one or more of the following:
1. The sequence context, e.g., a subject interval to be evaluated or a nucleotide position therein, that is associated with a propensity for misalignment of reads. For example, the existence of a sequence element in or near the subject interval to be evaluated that is repeated elsewhere in the genome can cause misalignment and thereby reduce sequencing performance. Performance can be enhanced by selecting an alignment algorithm or an algorithm parameter that minimizes misalignment. In this case, the value for the alignment selector can be a function of the sequence context, e.g., the presence or absence of a sequence of length that is repeated at least a given number of times in the genome (or in the portion of the genome being analyzed).
2. The tumor type being analyzed. For example, a specific tumor type can be characterized by an increased rate of deletions. Thus, performance can be enhanced by selecting an alignment algorithm or algorithm parameter that is more sensitive to indels. In this case, the value for the alignment selector can be a function of the tumor type, e.g., an identifier for the tumor type. In some instances, the value is the identity of the tumor type, e.g., a solid tumor or a hematologic malignancy (or premaligancy).
3. The gene, type of gene, or genetic locus being analyzed. Oncogenes, by way of example, are often characterized by substitutions or in-frame indels. Thus, performance can be enhanced by selecting an alignment algorithm or algorithm parameter that is particularly sensitive to these variants and specific against other types of variants. Tumor suppressors are often characterized by frame-shift indels. Thus, sequencing performance can be enhanced by selecting an alignment algorithm or algorithm parameter that is particularly sensitive to these variants. Thus, performance can be enhanced by selecting an alignment algorithm or algorithm parameter matched with the subject interval to be evaluated. In this case, the value for the alignment selector can be a function of the gene, type of gene, or genetic locus, e.g., an identifier for gene, gene type, or microsatellite locus. In some instances, for example, the value is the identity of the gene.
4. The site (e.g., nucleotide position) being analyzed. In this case, the value for the alignment selector can be a function of the site or the type of site, e.g., an identifier for the site or site type. In an embodiment the value is the identity of the site. For example, if the gene containing the site is highly homologous with another gene, normal/fast short read alignment algorithms (e.g., BWA) may have difficulty distinguishing between the two genes, potentially necessitating more intensive alignment methods (e.g., Smith-Waterman) or even assembly (e.g., ARACHNE). Similarly, if the gene sequence contains low-complexity regions (e.g., AAAAAA), more intensive alignment methods may be necessary.
5. The variant, or type of variant, associated with the subject interval being evaluated, e.g., a substitution, insertion, deletion, translocation, or other rearrangement. Thus, performance can be enhanced by selecting an alignment algorithm or algorithm parameter that is more sensitive to the specific variant type. In this case, the value for the alignment selector can be a function of the type of variant, e.g., an identifier for the type of variant. In some instances, the value is the identity of the type of variant, e.g., a substitution.
6. The type of sample, e.g., a sample described herein. Sample type/quality can affect error (spurious observation of non-reference sequence) rates. Thus, performance can be enhanced by selecting an alignment algorithm or algorithm parameter that accurately models the true error rate in the sample. In this case the value for the alignment selector can be a function of the type of sample, e.g., an identifier for the sample type. In some instances, e.g., the value is the identity of the sample type.

Generally, the accurate detection of indel mutations is an exercise in alignment, as the spurious indel rate on the sequencing platforms described herein is relatively low (thus, even a handful of observations of correctly aligned indels can be strong evidence of mutation). Accurate alignment in the presence of indels can be difficult however (especially as indel length increases). In addition to the general issues associated with alignment, e.g., of substitutions, the indel itself can cause problems with alignment. For instance, a deletion of 2 bp of a dinucleotide repeat cannot be readily definitively placed. Both sensitivity and specificity can be reduced by incorrect placement of shorter (<15 bp) apparent indel-containing reads. Larger indels (e.g., getting closer in magnitude to the length of individual reads, e.g., reads of 36 bp) can cause failure to align the read at all, making detection of the indel impossible in the standard set of aligned reads.

Databases of cancer mutations can be used to address these problems and improve performance. To reduce false positive indel discovery (i.e., to improve specificity), regions around commonly expected indels can be examined for problematic alignments due to sequence context and addressed similarly to substitutions. To improve sensitivity of indel detection, several different approaches of using information on the indels expected in cancer can be used. For example, short-reads containing expected indels can be simulated and alignment attempted. The alignments can be studied and alignment parameters can be adjusted for problematic indel regions, for instance, by reducing gap open/extend penalties or by aligning partial reads (e.g. the first or second half of a read).

Alternatively, initial alignment can be attempted not just with the normal reference genome, but also with alternate versions of the genome containing each of the known or likely cancer indel mutations. In this approach, reads of indels that initially failed to align, or aligned incorrectly, are placed successfully on the alternate (mutated) version of the genome.

In this way, indel alignment (and thus mutation calling) can be optimized for the expected cancer genes/sites. As used herein, a sequence alignment algorithm embodies a computational method or approach used to identify the location within the genome from which a given read sequence (e.g., a short-read sequence from next-generation sequencing) most likely originated by assessing the similarity between the read sequence and a reference sequence. Any of a variety of algorithms can be applied to the sequence alignment problem. Some algorithms are relatively slow, but allow relatively high specificity. These include, e.g., dynamic programming-based algorithms. Dynamic programming is a method for solving complex problems by breaking them down into simpler steps. Other approaches are relatively more efficient, but are typically not as thorough. These include, e.g., heuristic algorithms and probabilistic methods designed for large-scale database search.

Alignment parameters are used in alignment algorithms to adjust the performance of an algorithm, e.g., to produce an optimal global or local alignment between a read sequence and a reference sequence. Alignment parameters can give weights for match, mismatch, and indels. For example, lower weights may allow alignments that include more mismatches and indels.

The sensitivity of alignment can be increased when an alignment algorithm is selected, or an alignment parameter is adjusted, based on tumor type, e.g., a tumor type that tends to have a particular mutation or mutation type.

The sensitivity of alignment can be increased when an alignment algorithm is selected, or an alignment parameter is adjusted, based on a particular gene or locus type (e.g., oncogene, tumor suppressor gene, microsatellite region). Mutations in different types of cancer-associated genes can have different impacts on cancer phenotype. For example, mutant oncogene alleles are typically dominant. Mutant tumor suppressor alleles are typically recessive, which means that in most cases both alleles of a tumor suppressor genes must be affected before an effect is manifested.

The sensitivity of alignment can be adjusted (e.g., increased) when an alignment algorithm is selected, or an alignment parameter is adjusted, based on mutation type (e.g., single nucleotide polymorphism, indel (insertion or deletion), inversion, translocation, tandem repeat).

The sensitivity of alignment can be adjusted (e.g., increased) when an alignment algorithm is selected, or an alignment parameter is adjusted, based on mutation site (e.g., a mutation hotspot). A mutation hotspot refers to a site in the genome where mutations occur up to 100 times more frequently than the normal mutation rate.

The sensitivity/specificity of alignment can be adjusted (e.g., increased) when an alignment algorithm is selected, or an alignment parameter is adjusted, based on sample type (e.g., cfDNA sample, ctDNA sample, FFPE sample, or CTC sample).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

In some instances, the mutation calling methods described herein may comprise one or more of:
(i) assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is based on a unique (as opposed to the other assignments) first and/or second values;
(ii) the assignment of method of (i), wherein at least 10, 20, 30, 40, 50, 60, 70, 80, 90, 100, 200, 300, 400, or 500 of the assignments are made with first values which are a function of a probability of a variant being present in less than 5, 10, or 20%, e.g., of the cells in a tumor type;
(iii) assigning a nucleotide value (e.g., calling a mutation) for at least X nucleotide positions, each of which of which being associated with a variant having a unique (as opposed to the other X−1 assignments) probability of being present in a tumor of type, e.g., the tumor type of said sample, wherein, optionally, each of said of X assignments is based on a unique (as opposed to the other X−1 assignments) first and/or second value (wherein X=2, 3, 5, 10, 20, 40, 50, 60, 70, 80, 90, 100, 200, 300, 400, or 500);
(iv) assigning a nucleotide value (e.g., calling a mutation) at a first and a second nucleotide position, wherein the likelihood of a first variant at said first nucleotide position being present in a tumor of type (e.g., the tumor type of said sample) is at least 2, 5, 10, 20, 30, or 40 times greater than the likelihood of a second variant at said second nucleotide position being present, wherein, optionally, each assignment is based on a unique (as opposed to the other assignments) first and/or second value;
(v) assigning a nucleotide value to a plurality of nucleotide positions (e.g., calling mutations), wherein said plurality comprises an assignment for variants falling into one or more, e.g., at least 3, 4, 5, 6, 7, or all, of the following probability percentage ranges: less than or equal to 0.01; greater than 0.01 and less than or equal to 0.02; greater than 0.02 and less than or equal to 0.03; greater than 0.03 and less than or equal to 0.04; greater than 0.04 and less than or equal to 0.05; greater than 0.05 and less than or equal to 0.1; greater than 0.1 and less than or equal to 0.2; greater than 0.2 and less than or equal to 0.5; greater than 0.5 and less than or equal to 1.0; greater than 1.0 and less than or equal to 2.0; greater than 2.0 and less than or equal to 5.0; greater than 5.0 and less than or equal to 10.0; greater than 10.0 and less than or equal to 20.0; greater than 20.0 and less than or equal to 50.0; and greater than 50 and less than or equal to 100.0%, wherein, a probability range is the range of probabilities that a variant at a nucleotide position will be present in a tumor type (e.g., the tumor type of said sample) or the probability that a variant at a nucleotide position will be present in the recited percentage (%) of the cells in a sample, a library from the sample, or library catch from that library, for a preselected type (e.g., the tumor type of said sample), and wherein, optionally, each assignment is based on a unique first and/or second value (e.g., unique as opposed to the other assignments in a recited probability range or unique as opposed to the first and/or second values for one or more or all of the other listed probability ranges);
(vi) assigning a nucleotide value (e.g., calling a mutation) for at least 1, 2, 3, 5, 10, 20, 40, 50, 60, 70, 80, 90, 100, 200, 300, 400, 500, 600, 700, 800, 900, or 1,000 nucleotide positions each, independently, having a variant present in less than 50, 40, 25, 20, 15, 10, 5, 4, 3, 2, 1, 0.5, 0.4, 0.3, 0.2, or 0.1% of the DNA in said sample, wherein, optionally, each assignment is based on a unique (as opposed to the other assignments) first and/or second value;
(vii) assigning a nucleotide value (e.g., calling a mutation) at a first and a second nucleotide position, wherein the likelihood of a variant at the first position in the DNA of said sample is at least 2, 5, 10, 20, 30, or 40 times greater than the likelihood of a variant at said second nucleotide position in the DNA of said sample, wherein, optionally, each assignment is based on a unique (as opposed to the other assignments) first and/or second value;
(viii) assigning a nucleotide value (e.g., calling a mutation) in one or more or all of the following:
(1) at least 1, 2, 3, 4 or 5 nucleotide positions having a variant present in less than 1% of the cells in said sample, of the nucleic acids in a library from said sample, or the nucleic acid in a library catch from that library;
(2) at least 1, 2, 3, 4 or 5 nucleotide positions having a variant present in 1-2% of the cells in said sample, of the nucleic acid in a library from said sample, or the nucleic acid in a library catch from that library;
(3) at least 1, 2, 3, 4 or 5 nucleotide positions having a variant present in greater than 2% and less than or equal to 3% of the cells in said sample, of the nucleic acid in a library from said sample, or the nucleic acid in a library catch from that library
(4) at least 1, 2, 3, 4 or 5 nucleotide positions having a variant present in greater than 3% and less than or equal to 4% of the cells in said sample, of the nucleic acid in a library from said sample, or the nucleic acid in a library catch from that library;
(5) at least 1, 2, 3, 4 or 5 nucleotide positions having a variant present in greater than 4% and less than or equal to 5% of the cells in said sample, of the nucleic acid in a library from said sample, or the nucleic acid in a library catch from that library;
(6) at least 1, 2, 3, 4 or 5 nucleotide positions having a variant present in greater than 5% and less than or equal to 10% of the cells in said sample, of the nucleic acid in a library from said sample, or the nucleic acid in a library catch from that library;
(7) at least 1, 2, 3, 4 or 5 nucleotide positions having a variant present in greater than 10% and less than or equal to 20% of the cells in said sample, of the nucleic acid in a library from said sample, or the nucleic acid in a library catch from that library;
(8) at least 1, 2 3, 4 or 5 nucleotide positions having a variant present in greater than 20% and less than or equal to 40% of the cells in said sample, of the nucleic acid in a library from said sample, or the nucleic acid in a library catch from that library;
(9) at least 1, 2 3, 4 or 5 nucleotide positions having a variant present at greater than 40% and less than or equal to 50% of the cells in said sample, of the nucleic acid in a library from said sample, or the nucleic acid in a library catch from that library; or
(10) at least 1, 2 3, 4 or 5 nucleotide positions having a variant present in greater than 50% and less than or equal to 100% of the cells in said sample, of the nucleic acid in a library from said sample, or the nucleic acid in a library catch from that library;
wherein, optionally, each assignment is based on a unique first and/or second value (e.g., unique as opposed to the other assignments in the recited range (e.g., the range in (1) of less than 1%) or unique as opposed to a first and/or second values for a determination in one or more or all of the other listed ranges); or
(ix) assigning a nucleotide value (e.g., calling a mutation) at each of X nucleotide positions, each nucleotide position, independently, having a likelihood (of a variant being present in the DNA of said sample) that is unique as compared with the likelihood for a variant at the other X−1 nucleotide positions, wherein X is equal to or greater than 1, 2, 3, 5, 10, 20, 40, 50, 60, 70, 80, 90, 100, 200, 300, 400, 500, 600, 700, 800, 900, or 1,000, and wherein each assignment is based on a unique (as opposed to the other assignments) first and/or second value.

In some instances, a “threshold value” is used to evaluate reads, and select from the reads a value for a nucleotide position, e.g., calling a mutation at a specific position in a gene. In some instances, a threshold value for each of a number of subject intervals is customized or fine-tuned. The customization or tuning can be based on one or more of the factors described herein, e.g., the type of cancer in a sample, the gene in which the subject interval (subgenomic interval or expressed subgenomic interval) to be sequenced is located, or the variant to be sequenced. This provides for calling that is finely tuned to each of a number of subject intervals to be sequenced. In some instances, the method is particularly effective when a relatively large number of diverse subgenomic intervals are analyzed.

Thus, in another embodiment, the method comprises the following mutation calling method:
(i) acquiring, for each of said X subject intervals, a threshold value, wherein each of said acquired X threshold values is unique as compared with the other X−1 threshold values, thereby providing X unique threshold values;
(ii) for each of said X subject intervals, comparing an observed value which is a function of the number of reads having a nucleotide value at a nucleotide position with its unique threshold value, thereby applying to each of said X subject intervals its unique threshold value; and
(iii) optionally, responsive to the result of said comparison, assigning a nucleotide value to a nucleotide position, wherein X is equal to or greater than 2.

In some instances, the method includes assigning a nucleotide value to at least 2, 3, 5, 10, 20, 40, 50, 60, 70, 80, 90, 100, 200, 300, 400, 500, 600, 700, 800, 900, or 1,000 nucleotide positions, each having, independently, a first value that is a function of a probability that is less than 0.5, 0.4, 0.25, 0.15, 0.10, 0.05, 0.04, 0.03, 0.02, or 0.01.

In some instances, the method includes assigning a nucleotide value to at each of at least X nucleotide positions, each independently having a first value that is unique as compared with the other X−1 first values, and wherein each of said X first values is a function of a probability that is less than 0.5, 0.4, 0.25, 0.15, 0.10, 0.05, 0.04, 0.03, 0.02, or 0.01, wherein X is equal to or greater than 1, 2, 3, 5, 10, 20, 40, 50, 60, 70, 80, 90, 100, 200, 300, 400, 500, 600, 700, 800, 900, or 1,000.

In some instances, a nucleotide position in at least 20, 40, 60, 80, 100, 120, 140, 160 or 180, 200, 300, 400, 500, 600, 700, 800, 900, 1,000, 2,000, 3,000, 4,000, or 5,000 genes or genomic loci (e.g., microsatellite loci) is assigned a nucleotide value. In some instances, unique first and/or second values are applied to subject intervals in each of at least 10%, 20%, 30%, 40%, or 50% of said genes or genomic loci analyzed.

In some instances, the method may comprise optimizing threshold values for a relatively large number of subject intervals, as is seen, e.g., in the following embodiments.

In some instances, a unique threshold value is applied to subject intervals, e.g., subgenomic intervals or expressed subgenomic intervals, in each of at least 3, 5, 10, 20, 40, 50, 60, 70, 80, 90, 100, 200, 300, 400, 500, 600, 700, 800, 900, 1,000, 2,000, 3,000, 4,000, or 5,000 different genes or genomic loci.

In some instances, a nucleotide position in at least 20, 40, 60, 80, 100, 120, 140, 160 or 180, 200, 300, 400, 500, 1,000, 2,000, 3,000, 4,000, or 5,000 genes or genomic loci is assigned a nucleotide value. In some instances, a unique threshold value is applied to a subject interval in each of at least 10%, 20%, 30%, 40%, or 50% of said genes or genomic loci analyzed.

In some instances, at least 10%, 20%, 30%, 40%, 50%, 60%, 70%, 80%, or 90% of the mutation calls made using the method are for subject intervals from genes or genomic loci described herein. In some instances, at least 10%, 20%, 30%, 40%, 50%, 60%, 70%, 80%, or 90% of the unique threshold values described herein are for subject intervals from genes or genomic loci described herein. In some instances, at least 10%, 20%, 30%, 40%, 50%, 60%, 70%, 80%, or 90% of the mutation calls annotated, or reported, e.g., to a third party, are for subject intervals from genes or genomic loci.

In some instances, the assigned value for a nucleotide position is transmitted to a third party, optionally, with explanatory annotation. In some instances, the assigned value for a nucleotide position is not transmitted to a third party. In some instances, the assigned value for a plurality of nucleotide positions are transmitted to a third party, optionally, with explanatory annotations, and the assigned value for a second plurality of nucleotide positions are not transmitted to a third party.

In some instances, the method comprises assigning one or more reads to a subject, e.g., by barcode deconvolution.

In some instances, the method comprises assigning one or more reads as a tumor read or a control read, e.g., by barcode deconvolution. In some instances, the method comprises mapping, e.g., by alignment with a reference sequence, each of said one or more reads. In an embodiment, the method comprises memorializing a called mutation.

In some instances, the method comprises annotating a called mutation, e.g., annotating a called mutation with an indication of mutation structure, e.g., a missense mutation, or function, e.g., a disease phenotype. In some instances, the method comprises acquiring nucleotide sequence reads for tumor and control nucleic acid. In some instances, the method comprises calling a nucleotide value, e.g., a variant (e.g., a mutation), for each of the subject intervals (e.g., subgenomic intervals, expressed subgenomic intervals, or both), e.g., using a Bayesian calling method or a non-Bayesian calling method. In some instances, the method comprises evaluating a plurality of reads that include at least one SNP. In some instances, the method comprises determining a SNP allele ratio in the sample and/or control reads.

In some instances, the method further comprises building a database of sequencing/alignment artifacts for the targeted subgenomic regions. In some instances, the database can be used to filter out spurious mutation calls and improve specificity. In some instances, the database is built by sequencing unrelated samples or cell-lines, and recording non-reference allele events that appear more frequently than expected due to random sequencing error alone in one or more normal samples. This approach may classify germline variations as artifact, but that may be acceptable in a method concerned with somatic mutations. The misclassification of germline variation as artifact may be ameliorated if desired by filtering the database for known germline variations (removing common variants) and for artifacts that appear in only single individuals (removing rarer variations).

In some instances, the next-generation sequencing methods combined with alignment and/or mutation calling algorithms described herein can detect variant alleles present at an allele frequency of less than 20%, less than 10%, less than 5%, less than 4%, less than 3%, less than 2%, or less than 1% in a sample.

In some instances, the disclosed methods comprise assigning one or more reads to a subject, e.g., by barcode deconvolution. In some instances, the disclosed methods comprise assigning one or more reads as a tumor read or a control read, e.g., by barcode deconvolution.

In some instances, the method comprises mapping, e.g., by alignment with a reference sequence, each of said one or more reads. In some instances, the method comprises memorializing a called mutation. In some instances, the method comprises annotating a called mutation, e.g., annotating a called mutation with an indication of mutation structure, e.g., a missense mutation, or function, e.g., a disease phenotype.

In an embodiment, the method comprises acquiring nucleotide sequence reads for tumor and control nucleic acid. In some instances, the method comprises calling a nucleotide value, e.g., a variant, e.g., a mutation, for each of the subject intervals (e.g., sub genomic intervals, expressed subgenomic intervals, or both), e.g., using a Bayesian calling method or a non-Bayesian calling method.

In some instances, the method comprises evaluating a plurality of reads that include at least one SNP. In some instances, the method comprises determining an SNP allele ratio in the sample and/or control read.

In some instances, the method further comprises building a database of sequencing/alignment artifacts for the targeted subgenomic regions. In some instances, the database can be used to filter out spurious mutation calls and improve specificity. In some instances, the database is built by sequencing unrelated samples or cell-lines and recording non-reference allele events that appear more frequently than expected due to random sequencing error alone in one or more of these normal samples. This approach may classify germline variation as artifact, but that may be acceptable in a method concerned with somatic mutations. This misclassification of germline variation as artifact may be ameliorated if desired by filtering the database for known germline variations (e.g., removing common variants) and for artifacts that appear in only one individual (e.g., removing rare variants).

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for detecting and classifying reversion mutations in a sample from a subject. The systems may comprise, e.g., one or more processors, and a memory unit communicatively coupled to the one or more processors and configured to store instructions that, when executed by the one or more processors, cause the system to: receive sequence data for nucleic acid sequences that reside within one or more gene loci within a subgenomic interval in a sample from a subject; identify a gene locus of the one or more gene loci for which the gene locus comprises two or more variant sequences; categorize the two or more variant sequences in the gene locus according to a structural feature or functional effect; compare the structural features or functional effects of the two or more categorized variant sequences in the gene locus; and classify the two or more categorized variant sequences, based on the comparison, wherein the classification indicates whether the two or more categorized variant sequences comprise a reversion mutation.

In some instances, the disclosed systems may further comprise a sequencer, e.g., a next generation sequencer (also referred to as a massively parallel sequencer).

In some instances, the disclosed systems may be used for detection of reversion mutations in any of a variety of samples as described herein (e.g., a tissue sample, biopsy sample, hematological sample, or liquid biopsy sample derived from the subject).

In some instances, the plurality of gene loci for which sequencing data is processed to detect and classify reversion mutations may comprise at least 1, 2, 3, 4, 5, 6, 7, 8, 9, 10, or more than 10 gene loci.

In some instance, the nucleic acid sequence data is acquired using a next generation sequencing (NGS) technique (also referred to as a massively parallel sequencing (MPS) technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the detection and classification of reversion mutations is used to select, initiate, adjust, or terminate a treatment for cancer in the subject (e.g., a patient) from which the sample was derived, as described elsewhere herein.

In some instances, the disclosed systems may further comprise a next generation (or massively parallel) sequencing platform (e.g., a Roche 454, Illumina Solexa, ABI-SOLiD, ION Torrent, or Pacific Bioscience sequencing platform), sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

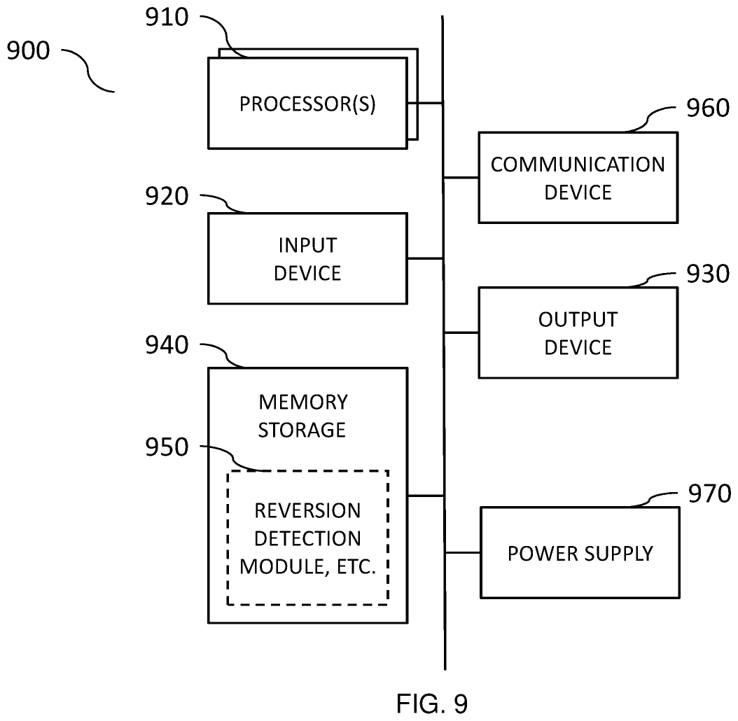

FIG. 9 illustrates an example of a computing device or system in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, and communication devices 960, and power supplies 970. Software 950 residing in memory or storage device 940 may comprise, e.g., an operating system as well as software for executing the methods described herein. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

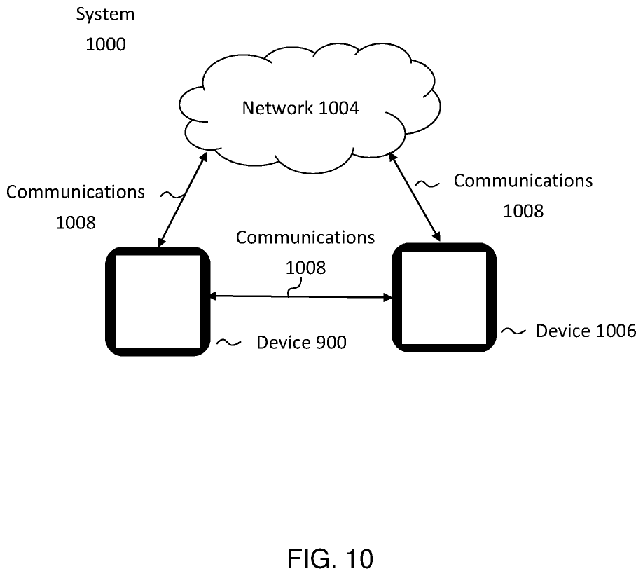

Device 900 may be connected to a network (e.g., network 1004, as shown in FIG. 10 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a power supply 970, which can be any suitable power supply.

FIG. 10 illustrates an example of a computing system in accordance with one embodiment. In system 1000, device 900 (e.g., as described above and illustrated in FIG. 9) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, Devices 900 and 1006 can communicate directly (instead of, or in addition to, communicating via network 604), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

### EXAMPLES

### Example 1—Examples of Type 1, Type 2, and Type 3 Reversion Mutations

A Type 1 reversion mutation in the present non-limiting example refers to a missense mutation that occurs in the same codon as a deleterious mutation (e.g., a nonsense mutation, missense mutation, or frameshift mutation) and reverses the impact of the deleterious mutation. Examples of Type 1 reversion mutations include, but are not limited to, a missense mutation that corrects a missense or nonsense mutation, a missense mutation that corrects a frameshift incurred by an insertion, and a missense mutation that corrects a frameshift incurred by a deletion.

Table 2 provides a non-limiting example of a Type 1 reversion mutation in the BRCA1 gene, along with the respective allele frequencies and sequencing coverage for the primary and secondary mutations. A c.1045G>T base substitution (i.e., a nonsense mutation comprising the substitution of a T for a G at nucleotide position number 1045 relative to the first nucleotide of the start codon for translation) introduces a stop codon, and would result in the expression of a truncated protein, p.E349* (i.e., a truncated protein in which the glutamate residue at amino acid position 349 is replaced by the stop codon), if not countered by a secondary mutation. The missense mutation c.1046A>G occurs within the same amino acid codon as the nonsense mutation, and results in the expression of a functional mutant p.E349G protein (i.e., a mutated protein in which the glutamate residue at amino acid position 349 is replaced by a glycine residue).

TABLE 2
Example of Type 1 Reversion Mutation

| | | | Allele | |
|---|---|---|---|---|
| Gene | CDS | Protein | frequency | Depth |
| BRCA1 | 1045G > T | E349* | 21.25 | 2560 |
| BRCA1 | 1046A > G | E349G | 8.69 | 2554 |

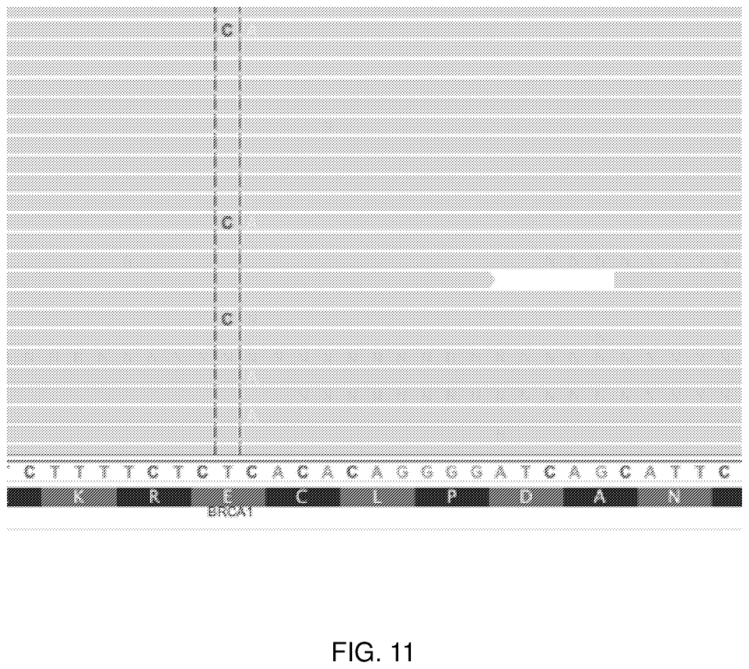

FIG. 11 provides an example of the Type 1 reversion mutation listed in Table 2 as viewed in the graphical user interface (GUI) of a genomics data visualization tool (e.g., the Integrative Genomics Viewer (IGV) (Broad Institute, Cambridge, MA)). The original deleterious mutation in the BRCA 1 gene locus introduces a stop codon due to the c.1045G>T base substitution (shown as the green ‘A’ in the horizontal gray rows representing aligned sequence reads) and, on its own, would result in the expression of the truncated protein, p.E349*, as noted above. The missense mutation c.1046A>G (shown as a blue ‘C’ in the horizontal gray rows representing sequence reads) occurs within the same amino acid codon as the nonsense mutation. The presence of the reversion mutation is evidenced by the appearance of both mutations in the same sequence read.

A Type 2 reversion mutation refers to a non-frameshift deletion mutation that occurs in the same gene as a deleterious mutation and reverses the impact of the deleterious mutation. Table 3 provides a non-limiting example of a Type 2 reversion mutation in the BRCA 1 gene. A c.1175_1214del40 mutation (i.e., a deletion mutation comprising the deletion of nucleotides 1175 through 1214) that would result in the expression of a frameshifted mutant protein, p.L392fs*5 (i.e., a mutant frameshifted protein in which the leucine residue at position 392 is altered and a stop codon is introduced 5 amino acids away from amino acid 392 as a result of deleting nucleotides 1175 through 1214), if not countered by a secondary mutation. The occurrence of the non-frameshift deletion mutation c.1129_1251del123 in the same gene results in the expression of a mutant protein, p.S377_N417del (i.e., a mutated protein in which the amino acid residues from serine 377 through asparagine 417 have been deleted).

TABLE 3
Example of Type 2 Reversion Mutation

| | | | Allele | |
|---|---|---|---|---|
| Gene | CDS | Protein | frequency | Depth |
| BRCA1 | 1175_1214del40 | L392fs*5 | 68.49 | 991 |
| BRCA1 | 1129_1251del123 | S377_N417del | 31.58 | 1388 |

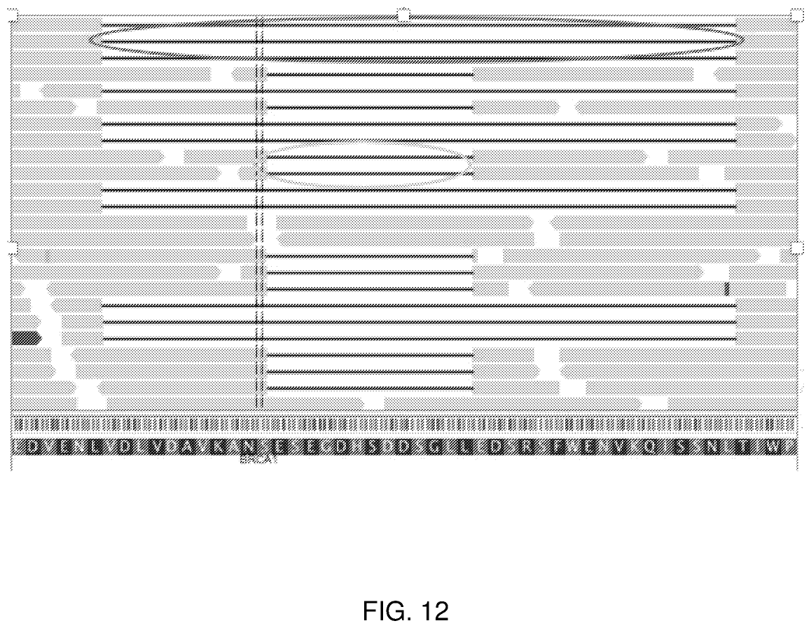

FIG. 12 provides an example of the Type 2 reversion mutation listed in Table 3 as viewed in the GUI of a genomics data visualization tool. The non-frameshift deletion (example highlighted in red) encompasses the original deleterious frameshift mutation (example highlighted in yellow).

A Type 3 reversion mutation refers to a second frameshift indel that restores the open reading frame that was disrupted by a first frameshift indel. Table 4 provides a non-limiting example of a Type 3 reversion mutation in the BRCA2 gene. A c.3770_3771delAG frameshift indel (i.e. a frameshift deletion comprising the deletion of A and G at nucleotide positions 3770 and 3771) would result in the expression of a frameshifted mutant protein, p.E1257fs*9 (i.e., a mutated protein in which the glutamate residue at position 1257 is the first altered amino acid residue and the length of the frame shift is 9 amino acid residues, including a stop codon), if not countered by a secondary mutation. The occurrence of the c.3756_3759delGTCT indel (i.e., a frameshift deletion comprising the deletion of G, T, C, and T at nucleotide positions 3756, 3757, 3758, and 3759, respectively) in the same gene results in the expression of a mutant protein, p.S1253fs*10 (i.e., a mutated protein in which the serine residue at position 1253 is the first altered amino acid residue and the length of the frame shift is 10 amino acid residues, including a stop codon).

TABLE 4
Example of Type 3 Reversion Mutation

| | | | Allele | |
|---|---|---|---|---|
| Gene | CDS | Protein | frequency | Depth |
| BRCA2 | 3770_3771delAG | E1257fs*9 | 85.94 | 1693 |
| BRCA2 | 3756_3759delGTCT | S1253fs*10 | 11.46 | 1737 |

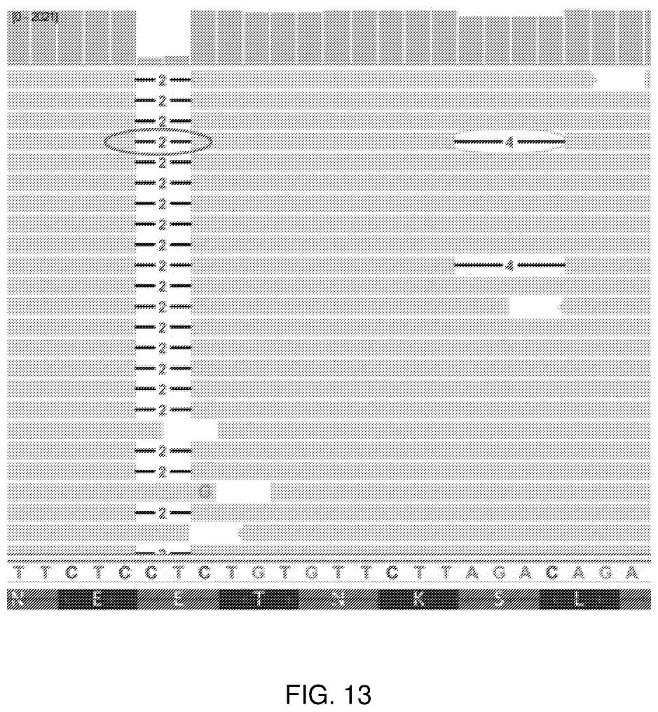

FIG. 13 provides an example of the Type 3 reversion mutation listed in Table 4 as viewed in the GUI of a genomics data visualization tool. The first frameshift deletion (example highlighted in red) is corrected by the second frameshift deletion (example highlighted in yellow). The net loss in bases is six, resulting in a net non-frameshift deletion.

Table 5 and Table 6 provide non-limiting examples of missense mutations that are often found in the BRCA1 and BRCA 2 genes, respectively.

TABLE 5
Deleterious BRCA1 missense mutations

| | | | | | | | | | | |
|---|---|---|---|---|---|---|---|---|---|---|
| M1V | I26N | H41R | C47F | C64Y | S770L | T1685A | R1699W | S1722F | I1766N | G1788V |
| M1T | T37K | C44S | C61S | C64W | R1495T | T1685I | R1699Q | V1736A | I1766S | P1812A |
| M1R | C39R | C44Y | C61G | R71G | R1495M | D1692N | G1706R | G1738R | G1770V | A1823T |
| M1I | C39G | C44F | C61Y | R71K | R1495K | M1689R | G1706E | G1738E | M1775K | V1833M |
| M18T | C39Y | C47S | C64R | R71T | E1559K | D1692H | A1708E | K1759N | M1775R | W1837R |
| L22S | C39W | C47Y | C64G | R71M | E1559Q | D1692Y | S1715R | L1764P | C1787S | V1838E |

TABLE 6
Deleterious BRCA2 missense mutations

| | | | | | | | |
|---|---|---|---|---|---|---|---|
| M1V | D23Y | V211L | R2336H | R2659T | T2722K | G2748D | D3095E |
| M1T | S142N | Y600C | T2412I | R2659K | T2722R | A2911E | N3124I |
| M1R | S142I | K1530N | R2602T | E2663V | D2723H | E3002K | N3187K |
| M1I | V159M | R2336P | W2626C | S2670L | D2723G | R3052W | |
| D23N | V211I | R2336L | I2627F | I2675V | G2724W | D3095G | |

### Exemplary Embodiments

Exemplary embodiments of the disclosed methods and systems include:
1. A method comprising:
   providing a plurality of nucleic acid molecules obtained from a sample from a subject;
   ligating one or more adapters onto one or more nucleic acid molecules from the plurality of nucleic acid molecules;
   amplifying nucleic acid molecules from the plurality of nucleic acid molecules;
   capturing nucleic acid molecules from the amplified nucleic acid molecules;
   sequencing, using a sequencer, the captured nucleic acid molecules to obtain a plurality of sequence reads that represent the captured nucleic acid molecules, wherein one or more of the plurality of sequencing reads overlap one or more gene loci within a subgenomic interval in the sample;
   receiving, at one or more processors, sequence read data for the plurality of sequence reads;
   identifying, using the one or more processors, a gene locus of the one or more gene loci for which the gene locus comprises two or more variant sequences;
   categorizing, using the one or more processors, the two or more variant sequences in the gene locus according to one or more attributes of the two or more variant sequences;
   comparing, using the one or more processors, the one or more attributes of the two or more categorized variant sequences in the gene locus;
   classifying, using the one or more processors, the two or more categorized variant sequences in the gene locus based on the comparison, wherein the classification indicates whether the two or more categorized variant sequences comprise a reversion mutation; and
   identifying the reversion mutation in a genomic profile associated with the subject.
2. The method of embodiment 1, wherein the subject is suspected of having or is determined to have cancer.
3. The method of embodiment 1 or embodiment 2, further comprising obtaining the sample from the subject.
4. The method of any one of embodiments 1 to 3, wherein the sample comprises a tissue biopsy sample, a liquid biopsy sample, or a normal control.
5. The method of embodiment 4, wherein the sample is a liquid biopsy sample and comprises blood, plasma, cerebrospinal fluid, sputum, stool, urine, or saliva.
6. The method of embodiment 4, wherein the sample is a liquid biopsy sample and comprises circulating tumor cells (CTCs).
7. The method of embodiment 4, wherein the sample is a liquid biopsy sample and comprises cell-free DNA (cfDNA), circulating tumor DNA (ctDNA), or any combination thereof.
8. The method of any one of embodiments 1 to 7, wherein the plurality of nucleic acid molecules comprises a mixture of tumor nucleic acid molecules and non-tumor nucleic acid molecules.
9. The method of embodiment 8, wherein the tumor nucleic acid molecules are derived from a tumor portion of a heterogeneous tissue biopsy sample, and the non-tumor nucleic acid molecules are derived from a normal portion of the heterogeneous tissue biopsy sample.
10. The method of embodiment 8, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
11. The method of any one of embodiments 1 to 10, wherein the one or more adapters comprise amplification primers, flow cell adapter sequences, substrate adapter sequences, or sample index sequences.
12. The method of any one of embodiments 1 to 11, wherein the captured nucleic acid molecules are capture from the amplified nucleic acid molecules by hybridization to one or more bait molecules.
13. The method of embodiment 12, wherein the one or more bait molecules comprise nucleic acid molecules, and wherein each bait molecule comprises a region that is complementary to a region of a captured nucleic acid molecule.
14. The method of any one of embodiments 1 to 13, wherein amplifying nucleic acid molecules comprises performing a polymerase chain reaction (PCR) amplification technique, a non-PCR amplification technique, or an isothermal amplification technique.
15. The method of any one of embodiments 1 to 14, wherein the sequencing comprises use of a massively parallel sequencing (MPS) technique, whole genome sequencing (WGS), whole exome sequencing, targeted sequencing, direct sequencing, or Sanger sequencing technique.
16. The method of embodiment 15, wherein the sequencing comprises use of a massively parallel sequencing (MPS) technique, and the massively parallel sequencing technique comprises next generation sequencing (NGS).
17. The method of any one of embodiments 1 to 16, wherein the sequencer comprises a massively parallel sequencer.
18. The method of any one of embodiments 1 to 17, wherein the one or more attributes of the two or more variant sequences comprise a structural feature or functional effect.
19. The method of embodiment 18, wherein a structural feature of a variant sequence comprises a substitution mutation (point mutation), an intragenic deletion, an insertion/deletion (indel) mutation, a splice site alteration, or a copy number alteration.
20. The method of embodiment 18 or embodiment 19, wherein a functional effect comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event.
21. The method of any one of embodiments 18 to 20, wherein the reversion mutation is classified, based on the comparison of the structural features or functional effects of the two or more categorized variant sequences in the gene locus, as: (i) a missense mutation that restores a deleterious mutation in the same codon, (ii) a non-frameshift deletion spanning a nucleic acid sequence region comprising a deleterious mutation in a gene locus, (iii) a second frameshift indel that restores the open reading frame that was disrupted by a first frameshift indel, (iv) a splice-site alteration occurring at a same exon as a deleterious mutation, (v) a copy number loss event occurring at a same exon as a deleterious mutation (vi) a synonymous mutation that occurs at a same amino acid position as a deleterious mutation, or (vii) an intragenic deletion event spanning a nucleic acid sequence region comprising a deleterious mutation.
22. The method of any one of embodiments 1 to 21, further comprising generating, by the one or more processors, a report indicating whether or not a reversion mutation in present in a gene locus of the one or more gene loci.
23. The method of embodiment 22, comprising transmitting the report to a healthcare provider.
24. The method of embodiment 23, wherein the report is transmitted via a computer network or a peer-to-peer connection.
25. A computer-implemented method comprising:
   receiving, at one or more processors, sequence read data for a plurality of sequence reads that overlap one or more gene loci within a subgenomic interval in a sample from a subject;
   identifying, using the one or more processors, a gene locus of the one or more gene loci for which the gene locus comprises two or more variant sequences;
   categorizing, using the one or more processors, the two or more variant sequences in the gene locus according to one or more attributes of the two or more variant sequences;
   comparing, using the one or more processors, the one or more attributes of the two or more categorized variant sequences in the gene locus; and
   classifying, using the one or more processors, the two or more categorized variant sequences in the gene locus based on the comparison, wherein the classification indicates whether the two or more categorized variant sequences comprise a reversion mutation.
26. The computer-implemented method of embodiment 25, further comprising identifying the reversion mutation in a genomic profile associated with the subject.
27. The computer-implemented method of embodiment 25 or embodiment 26, wherein the one or more attributes of the two or more variant sequences comprise a structural feature or functional effect.
28. The computer-implemented method of any one of embodiments 25 to 27, wherein the one or more gene loci comprise a tumor suppressor gene or fragment thereof, a homologous recombination repair gene or fragment thereof, or any combination thereof.
29. The computer-implemented method of any one of embodiments 25 to 28, wherein the one or more gene loci comprise APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof.
30. The computer-implemented method of any one of embodiments 27 to 29, wherein a structural feature of a variant sequence comprises a substitution mutation (point mutation), an intragenic deletion, an insertion/deletion (indel) mutation, a splice site alteration, or a copy number alteration.
31. The computer-implemented method of any one of embodiments 27 to 30, wherein a functional effect of a variant sequence comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event.
32. The computer-implemented method of embodiment 31, wherein the functional effect of a variant sequence comprises a frameshift mutation, wherein the frameshift mutation results from an insertion of one or more nucleotides, and wherein a total number of inserted nucleotides is not a multiple of 3.
33. The computer-implemented method of embodiment 31, wherein the functional effect of a variant sequence comprises a frameshift mutation, wherein the frameshift mutation results from a deletion of one or more nucleotides, and wherein a total number of deleted nucleotides is not a multiple of 3.
34. The computer-implemented method of any one of embodiments 27 to 33, wherein if a first variant sequence of the two or more variant sequences comprises a truncating event and a second variant sequence of the two or more variant sequences comprises a mutation that restores an open reading frame, a comparison of the structural features and functional effects of the truncating event and the open reading frame event is executed, and the reversion mutation is classified, based on the comparison, as: (i) a possible missense mutation that restores a deleterious mutation in the same codon, (ii) a possible non-frameshift deletion spanning a nucleic acid sequence region comprising a deleterious mutation in a gene locus, or (iii) a possible second frameshift indel that restores the open reading frame that was disrupted by a first frameshift indel.
35. The computer-implemented method of embodiment 34, wherein if the truncating event comprises a nonsense substitution mutation, the mutation that restores the open reading frame comprises a missense substitution mutation, and a starting base position and an ending base position for the nonsense substitution mutation are located at a same corresponding amino acid position as the missense substitution mutation, the reversion mutation is classified as a missense mutation that restores a deleterious mutation in the same codon.
36. The computer-implemented method of embodiment 34, wherein if the truncating event comprises a frameshift insertion mutation, the mutation that restores the open reading frame comprises a missense substitution mutation, and a starting base position and an ending base position for the missense substitution mutation are located at a same corresponding amino acid position as the frameshift insertion mutation, the reversion mutation is classified as a missense mutation that restores a deleterious mutation in the same codon.
37. The computer-implemented method of embodiment 34, wherein if the truncating event comprises a frameshift deletion mutation, the mutation that restores the open reading frame comprises a missense substitution mutation, and a starting base position and an ending base position for the missense substitution mutation are located at a same corresponding amino acid position as the frameshift deletion mutation, the reversion mutation is classified as a missense mutation that restores a deleterious mutation in the same codon.
38. The computer-implemented method of embodiment 34, wherein if the mutation that restores the open reading frame comprises a non-frameshift deletion mutation, and a starting base position and an ending base position for the truncating event are located between a starting base position and an ending base position of the non-frameshift deletion mutation, the reversion mutation is classified as a non-frameshift deletion spanning a nucleic acid sequence region comprising a deleterious mutation in the gene locus.
39. The computer-implemented method of embodiment 34, wherein if the mutation that restores an open reading frame comprises a non-frameshift deletion mutation, and the truncating mutation comprises an insertion mutation having a starting base position and an ending base position that are between a starting base position and an ending base position for the non-frameshift deletion mutation, the reversion mutation is classified as a non-frameshift deletion spanning a nucleic acid sequence region comprising a deleterious mutation in the gene locus.
40. The computer-implemented method of any one of embodiments 27 to 33, wherein if both a first variant sequence and a second variant sequence of the two or more categorized variant sequences comprise a truncating event, a comparison of the structural features or functional effects of the first truncating event and the second truncating event is executed, and the reversion mutation is classified, based on the comparison, as a possible second frameshift indel that restores an open reading frame that was disrupted by a first frameshift indel.
41. The computer-implemented method of embodiment 40, wherein if both the first truncating event and the second truncating event are indel mutations, the indel mutation corresponding to the second truncating event occurs at a position upstream from a stop codon for the indel mutation corresponding to the first truncating event, and if a net change in sequence length caused by the first and second truncating events is divisible by 3, the reversion mutation is classified as a second frameshift indel that restores an open reading frame that was disrupted by a first frameshift indel.
42. The computer-implemented method of any one of embodiments 27 to 33, wherein if a first variant sequence of the two or more variant sequences comprises a deleterious truncating event and a second variant sequence of the two or more variant sequences comprises a splice site alteration, a comparison of the structural features or functional effects of the deleterious truncating event and the splice site alteration is executed, and the reversion mutation is classified, based on the comparison, as a splice site alteration affecting the same exon as the deleterious truncation event.
43. The computer-implemented method of any one of embodiments 27 to 33, wherein if a first variant sequence of the two or more variant sequences comprises a deleterious truncating event and a second variant sequence of the two or more variant sequences comprises an intragenic deletion, and a starting base position and ending base position of the truncating event are between a starting base position and ending base position of the intragenic deletion, a comparison of the structural features or functional effects of the deleterious truncating event and the intragenic deletion is executed, and the reversion mutation is classified, based on the comparison, as the intragenic deletion spanning a nucleic acid sequence region comprising the deleterious truncating event.
44. The computer-implemented method of any one of embodiments 27 to 33, wherein if a first variant sequence of the two or more variant sequences comprises a truncating event and a second variant sequence of the two or more variant sequences comprises a copy number loss event, a comparison of the structural features or functional effects of the truncating event and the copy number loss event is executed, and the reversion mutation is classified, based on the comparison, as a possible copy number loss of a complete exon comprising the truncating event.
45. The computer-implemented method of embodiment 44, wherein if the truncating event has a starting base position and an ending base position located between a starting base position and an ending base position for the copy number loss event, and the copy number loss event comprises loss of a complete exon, the reversion mutation is classified as a copy number loss of a complete exon comprising the truncating event.
46. The computer-implemented method of any one of embodiments 27 to 33, wherein if a first variant sequence of the two or more variant sequences comprises a truncating event and a second variant sequence of the two or more variant sequences comprises a non-truncating event, a comparison of the structural features or functional effects of the truncating event and the non-truncating event is executed, and the reversion mutation is classified, based on the comparison, as a possible synonymous mutation occurring at the same amino acid position as a deleterious mutation.
47. The computer-implemented method of embodiment 46, wherein if the truncating event comprises a synonymous or nonsense mutation and the non-truncating event comprises a missense mutation, and wherein if the truncating and non-truncating events occur within a same codon, the reversion mutation is classified as a synonymous mutation occurring at the same amino acid position as a deleterious mutation.
48. The computer-implemented method of embodiment 46, wherein if the truncating event comprises a frameshift insertion mutation and the non-truncating event comprises a synonymous mutation, and wherein if the non-truncating synonymous mutation comprises an insertion that occurs within a same nucleotide range as the truncating frameshift insertion, the reversion mutation is classified as a synonymous mutation occurring at the same amino acid position as a deleterious mutation.
49. The computer-implemented method of embodiment 46, wherein if the truncating event comprises a frameshift deletion mutation and the non-truncating event comprises a synonymous mutation, and wherein if the non-truncating synonymous mutation comprises an insertion that occurs within a same nucleotide range as the truncating frameshift deletion, the reversion mutation is classified as a synonymous mutation occurring at the same amino acid position as a deleterious mutation.
50. The computer-implemented method of any one of embodiments 27 to 33, wherein if a first variant sequence of the two or more variant sequences comprises a truncating event and a second variant sequence of the two or more variant sequences comprises a splice site alteration, a comparison of the structural features or functional effects of the truncating event and the splice site alteration is executed, and the reversion mutation is classified, based on the comparison, as a possible intragenic deletion spanning a nucleic acid sequence region comprising a deleterious mutation.
51. The computer-implemented method of embodiment 50, wherein if the splice site alteration comprises an intragenic deletion that results in in-frame exon skipping of the exon in which the truncating event occurs, the reversion mutation is classified as an intragenic deletion spanning a nucleic acid sequence region comprising a deleterious mutation.
52. The computer-implemented method of any one of embodiments 25 to 51, further comprising selecting a cancer treatment for the subject based on the classification of the reversion mutation.
53. The computer-implemented method of embodiment 52, wherein the cancer treatment comprises a treatment for a cancer caused by mutations in one or more genes of a homologous recombination repair (HRR) pathway.
54. The computer-implemented method of embodiment 52 or embodiment 53, wherein the cancer treatment comprises a treatment for ovarian carcinoma, prostate carcinoma, or breast carcinoma.
55. The computer-implemented method of any one of embodiments 52 to 54, wherein the cancer treatment comprises use of a poly (ADP-ribose) polymerase inhibitor (PARPi), a platinum compound, a targeted immunotherapy treatment, or any combination thereof.
56. The computer-implemented method of any one of embodiments 25 to 55, wherein the detection and classification of the reversion mutation is performed without any manual curation of the sequence read data.
57. The computer-implemented method of any one of embodiments 25 to 56, further comprising obtaining the sample from the subject.
58. The computer-implemented method of any one of embodiments 25 to 57, wherein the sample comprises a tissue biopsy sample, a liquid biopsy sample, or a normal control.
59. The computer-implemented method of embodiment 58, wherein the sample is a liquid biopsy sample and comprises blood, plasma, cerebrospinal fluid, sputum, stool, urine, or saliva.
60. The computer-implemented method of embodiment 58, wherein the sample is a liquid biopsy sample and comprises circulating tumor cells (CTCs).
61. The computer-implemented method of embodiment 58, wherein the sample is a liquid biopsy sample and comprises cell-free DNA (cfDNA), circulating tumor DNA (ctDNA), or any combination thereof.
62. The computer-implemented method of any one of embodiments 25 to 61, further comprising extracting nucleic acid molecules from the sample.
63. The computer-implemented method of embodiment 62, wherein the nucleic acid molecules comprise tumor nucleic acid molecules.
64. The computer-implemented method of embodiment 62 or embodiment 63, wherein the nucleic acid molecules comprise non-tumor nucleic acid molecules.
65. The computer-implemented method of any one of embodiments 62 to 64, wherein nucleic acid molecules comprise deoxyribonucleic acid (DNA) molecules.
66. The computer-implemented method of embodiment 65, wherein the DNA molecules comprise cell-free DNA (cfDNA) molecules.
67. The computer-implemented method of embodiment 65 or embodiment 66, wherein the DNA molecules comprise circulating tumor DNA (ctDNA).
68. The computer-implemented method of any one of embodiments 62 to 65, wherein the nucleic acid molecules comprise ribonucleic acid (RNA) molecules.
69. The computer-implemented method of any one of embodiments 62 to 68, further comprising ligating one or more adapters to the nucleic acid molecules.
70. The computer-implemented method of any one of embodiments 62 to 69, further comprising amplifying the nucleic acid molecules.
71. The computer-implemented method of any one of embodiments 62 to 70, further comprising capturing a subset of nucleic acid molecules.
72. The computer-implemented method of embodiment 71, wherein the captured subset of nucleic acid molecules are captured by hybridization to one or more bait molecules.
73. The computer-implemented method of any one of embodiments 62 to 72, further comprising sequencing the captured nucleic acid molecules extracted from the sample to provide the sequence read data.
74. The computer-implemented method of embodiment 73, wherein the sequencing is performed using a massively parallel sequencer.
75. The computer-implemented method of embodiment 73 or embodiment 74, wherein the sequencing comprises targeted sequencing.
76. The computer-implemented method of embodiment 75, wherein the targeted sequencing comprises targeted sequencing of one or more genes associated with cancer, or portions thereof.
77. The computer-implemented method of embodiment 75 or embodiment 76, wherein the targeted sequencing comprises targeted sequencing of one or more exon regions.
78. The computer-implemented method of any one of embodiments 25 to 77, further comprising generating, by the one or more processors, a report that comprises a list of reversion mutations detected in the sample.
79. The computer-implemented method of embodiment 78, further comprising transmitting the report to a healthcare provider.
80. The computer-implemented method of embodiment 79, wherein the report is transmitted via a computer network or a peer-to-peer connection.
81. The computer-implemented method of any one of embodiments 26 to 80, wherein the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof.
82. The computer-implemented method of any one of embodiments 26 to 81, wherein the genomic profile for the subject further comprises results from a nucleic acid sequencing-based test.
83. The computer-implemented method of any one of embodiments 26 to 82, further comprising selecting an anti-cancer agent, administering an anti-cancer agent, or applying an anti-cancer treatment to the subject based on the genomic profile.
84. A method of selecting a treatment for a cancer in a subject, comprising:
   responsive to detecting and classifying a reversion mutation in a sample from the subject,
   selecting a cancer treatment for the subject, wherein the reversion mutation is identified and classified according to the method of any one of embodiments 25 to 80.
85. The method of embodiment 84, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
86. The method of embodiment 84 or embodiment 85, wherein the sample comprises a tissue biopsy sample, a liquid biopsy sample, or a normal control.
87. The method of embodiment 86, wherein the sample is a liquid biopsy sample and comprises blood, plasma, cerebrospinal fluid, sputum, stool, urine, or saliva.
88. The method of embodiment 86, wherein the sample is a liquid biopsy sample and comprises circulating tumor cells (CTCs).
89. The method of embodiment 86, wherein the sample is a liquid biopsy sample and comprises cell-free DNA (cfDNA), circulating tumor DNA (ctDNA), or any combination thereof.
90. The method of any one of embodiments 84 to 89, wherein the cancer treatment comprises a treatment for a cancer caused by mutations in one or more genes of a homologous recombination repair (HRR) pathway.
91. The method of any one of embodiments 84 to 90, wherein the cancer treatment comprises a treatment for ovarian carcinoma, prostate carcinoma, or breast carcinoma.
92. The method of any one of embodiments 84 to 91, wherein the cancer treatment comprises use of a poly (ADP-ribose) polymerase inhibitor (PARPi), a platinum compound, a targeted immunotherapy treatment, or any combination thereof.
93. A method of treating a cancer in a subject, comprising:
   responsive to detecting and classifying a reversion mutation in a sample from the subject, administering an effective amount of a cancer therapy to the subject, wherein the reversion mutation is identified and classified according to the method of any one of embodiments 25 to 80.
94. The method of embodiment 93, wherein the sample comprises a tissue biopsy sample, a liquid biopsy sample, or a normal control.
95. The method of embodiment 94, wherein the sample is a liquid biopsy sample and comprises blood, plasma, cerebrospinal fluid, sputum, stool, urine, or saliva.
96. The method of embodiment 94, wherein the sample is a liquid biopsy sample and comprises circulating tumor cells (CTCs).
97. The method of embodiment 94, wherein the sample is a liquid biopsy sample and comprises cell-free DNA (cfDNA), circulating tumor DNA (ctDNA), or any combination thereof.
98. The method of any one of embodiments 93 to 97, wherein the cancer treatment comprises a treatment for a cancer caused by mutations in one or more genes of a homologous recombination repair (HRR) pathway.
99. The method of any one of embodiments 93 to 98, wherein the cancer treatment comprises a treatment for ovarian carcinoma, prostate carcinoma, or breast carcinoma.
100. The method of any one of embodiments 93 to 99, wherein the cancer treatment comprises use of a poly (ADP-ribose) polymerase inhibitor (PARPi), a platinum compound, a targeted immunotherapy treatment, or any combination thereof.
101. A system comprising:
   one or more processors; and
   a memory communicatively coupled to the one or more processors and configured to store instructions that, when executed by the one or more processors, cause the system to:
      receive sequence read data for a plurality of sequence reads that overlap one or more gene loci within a subgenomic interval in a sample from a subject;
      identify a gene locus of the one or more gene loci for which the gene locus comprises two or more variant sequences;
      categorize the two or more variant sequences in the gene locus according to one or more attributes of the two or more variant sequences;
      compare the one or more attributes of the two or more categorized variant sequences in the gene locus; and
   classify the two or more categorized variant sequences, based on the comparison, wherein the classification indicates whether the two or more categorized variant sequences comprise a reversion mutation.
102. The system of embodiment 101, wherein the one or more attributes of the two or more variant sequences comprise a structural feature of functional effect.
103. The system of embodiment 101 or embodiment 102, further comprising a sequencer.
104. The system of embodiment 103, wherein the sequencer comprises a massively parallel sequencer.
105. The system of embodiment 104, wherein the massively parallel sequencer comprises a next generation sequencer.
106. The system of any one of embodiments 101 to 105, wherein the one or more gene loci comprise a tumor suppressor gene or fragment thereof, a homologous recombination repair gene or fragment thereof, or any combination thereof.
107. The system of any one of embodiments 101 to 106, wherein the one or more gene loci comprise APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof.
108. The system of any one of embodiments 102 to 107, wherein a structural feature of a variant sequence comprises a substitution mutation (point mutation), an intragenic deletion, an insertion/deletion (indel) mutation, a splice site alteration, or a copy number alteration.
109. The system of any one of embodiments 102 to 108, wherein a functional effect of a variant sequence comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event.
110. The system of any one of embodiments 102 to 109, wherein if a first variant sequence of the two or more variant sequences comprises a truncating event and a second variant sequence of the two or more variant sequences comprises a mutation that restores an open reading frame, a comparison of the structural features and functional effects of the truncating event and the open reading frame event is executed, and the reversion mutation is classified, based on the comparison, as: (i) a possible missense mutation that restores a deleterious mutation in the same codon, (ii) a possible non-frameshift deletion spanning a nucleic acid sequence region comprising a deleterious mutation in a gene locus, or (iii) a possible second frameshift indel that restores the open reading frame that was disrupted by a first frameshift indel.
111. The system of embodiment 110, wherein if the truncating event comprises a nonsense substitution mutation, the mutation that restores the open reading frame comprises a missense substitution mutation, and a starting base position and an ending base position for the nonsense substitution mutation are located at a same corresponding amino acid position as the missense substitution mutation, the reversion mutation is classified as a missense mutation that restores a deleterious mutation in the same codon.
112. The system of embodiment 110, wherein if the truncating event comprises a frameshift insertion mutation, the mutation that restores the open reading frame comprises a missense substitution mutation, and a starting base position and an ending base position for the missense substitution mutation are located at a same corresponding amino acid position as the frameshift insertion mutation, the reversion mutation is classified as a missense mutation that restores a deleterious mutation in the same codon.
113. The system of embodiment 110, wherein if the truncating event comprises a frameshift deletion mutation, the mutation that restores the open reading frame comprises a missense substitution mutation, and a starting base position and an ending base position for the missense substitution mutation are located at a same corresponding amino acid position as the frameshift deletion mutation, the reversion mutation is classified as a missense mutation that restores a deleterious mutation in the same codon.
114. The system of embodiment 110, wherein if the mutation that restores the open reading frame comprises a non-frameshift deletion mutation, and a starting base position and an ending base position for the truncating event are located between a starting base position and an ending base position of the non-frameshift deletion mutation, the reversion mutation is classified as a non-frameshift deletion spanning a nucleic acid sequence region comprising a deleterious mutation in the gene locus.
115. The system of embodiment 110, wherein if the mutation that restores an open reading frame comprises a non-frameshift deletion mutation, and the truncating mutation comprises an insertion mutation having a starting base position and an ending base position that are between a starting base position and an ending base position for the non-frameshift deletion mutation, the reversion mutation is classified as a non-frameshift deletion spanning a nucleic acid sequence region comprising a deleterious mutation in the gene locus.
116. The system of any one of embodiments 102 to 109, wherein if both a first variant sequence and a second variant sequence of the two or more categorized variant sequences comprise a truncating event, a comparison of the structural features or functional effects of the first truncating event and the second truncating event is executed, and the reversion mutation is classified, based on the comparison, as a possible second frameshift indel that restores an open reading frame that was disrupted by a first frameshift indel.
117. The system of embodiment 116, wherein if both the first truncating event and the second truncating event are indel mutations, the indel mutation corresponding to the second truncating event occurs at a position upstream from a stop codon for the indel mutation corresponding to the first truncating event, and if a net change in sequence length caused by the first and second truncating events is divisible by 3, the reversion mutation is classified as a second frameshift indel that restores an open reading frame that was disrupted by a first frameshift indel.
118. The system of any one of embodiments 102 to 109, wherein if a first variant sequence of the two or more variant sequences comprises a deleterious truncating event and a second variant sequence of the two or more variant sequences comprises a splice site alteration, a comparison of the structural features or functional effects of the deleterious truncating event and the splice site alteration is executed, and the reversion mutation is classified, based on the comparison, as a splice site alteration affecting the same exon as the deleterious truncation event.
119. The system of any one of embodiments 102 to 109, wherein if the first variant sequence of the two or more variant sequences comprises a deleterious truncating event and a second variant sequence of the two or more variant sequences comprises an intragenic deletion, and a starting base position and ending base position of the truncating event is between a starting base position and ending base position of the intragenic deletion, a comparison of the structural features or functional effects of the deleterious truncating event and the intragenic deletion is executed, and the reversion mutation is classified, based on the comparison, as the intragenic deletion spanning a nucleic acid sequence region comprising the deleterious truncating event.
120. The system of any one of embodiments 102 to 109, wherein if a first variant sequence of the two or more variant sequences comprises a truncating event and a second variant sequence of the two or more variant sequences comprises a copy number loss event, a comparison of the structural features or functional effects of the truncating event and the copy number loss event is executed, and the reversion mutation is classified, based on the comparison, as a possible copy number loss of a complete exon comprising the truncating event.
121. The system of embodiment 120, wherein if the truncating event has a starting base position and an ending base position located between a starting base position and an ending base position for the copy number loss event, and the copy number loss event comprises loss of a complete exon, the reversion mutation is classified as a copy number loss of a complete exon comprising the truncating event.
122. The system of any one of embodiments 102 to 109, wherein if a first variant sequence of the two or more variant sequences comprises a truncating event and a second variant sequence of the two or more variant sequences comprises a non-truncating event, a comparison of the structural features or functional effects of the truncating event and the non-truncating event is executed, and the reversion mutation is classified, based on the comparison, as a possible synonymous mutation occurring at the same amino acid position as a deleterious mutation.
123. The system of embodiment 122, wherein if the truncating event comprises a synonymous or nonsense mutation and the non-truncating event comprises a missense mutation, and wherein if the truncating and non-truncating events occur within a same codon, the reversion mutation is classified as a synonymous mutation occurring at the same amino acid position as a deleterious mutation.
124. The system of embodiment 122, wherein if the truncating event comprises a frameshift insertion mutation and the non-truncating event comprises a synonymous mutation, and wherein if the non-truncating synonymous mutation comprises an insertion that occurs within a same nucleotide range as the truncating frameshift insertion, the reversion mutation is classified as a synonymous mutation occurring at the same amino acid position as a deleterious mutation.
125. The system of embodiment 122, wherein if the truncating event comprises a frameshift deletion mutation and the non-truncating event comprises a synonymous mutation, and wherein if the non-truncating synonymous mutation comprises an insertion that occurs within a same nucleotide range as the truncating frameshift deletion, the reversion mutation is classified as a synonymous mutation occurring at the same amino acid position as a deleterious mutation.
126. The system of any one of embodiments 102 to 109, wherein if a first variant sequence of the two or more variant sequences comprises a truncating event and a second variant sequence of the two or more variant sequences comprises a splice site alteration, a comparison of the structural features or functional effects of the truncating event and the splice site alteration is executed, and the reversion mutation is classified, based on the comparison, as a possible intragenic deletion spanning a nucleic acid sequence region comprising a deleterious mutation.
127. The system of embodiment 126, wherein if the splice site alteration comprises an intragenic deletion that results in in-frame exon skipping of the exon in which the truncating event occurs, the reversion mutation is classified as an intragenic deletion spanning a nucleic acid sequence region comprising a deleterious mutation.
128. A non-transitory computer-readable storage medium storing one or more programs, the one or more programs comprising instructions, which when executed by one or more processors of a computer system, cause the computer system to:
   receive sequence read data for a plurality of sequence reads that overlap one or more gene loci within a subgenomic interval in a sample from a subject;
   identify a gene locus of the one or more gene loci for which the gene locus comprises two or more variant sequences;
   categorize the two or more variant sequences in the gene locus according to one or more attributes of the two or more variant sequences;
   compare the one or more attributes of the two or more categorized variant sequences in the gene locus; and
   classify the two or more categorized variant sequences, based on the comparison, wherein the classification indicates whether the two or more categorized variant sequences comprise a reversion mutation.
129. The non-transitory computer-readable storage medium of embodiment 128, wherein the one or more attributes of the two or more variant sequences comprise a structural feature or functional effect.
130. The non-transitory computer-readable storage medium of embodiment 128 or embodiment 129, wherein the one or more gene loci comprise a tumor suppressor gene or fragment thereof, a homologous recombination repair gene or fragment thereof, or any combination thereof.
131. The non-transitory computer-readable storage medium of any one of embodiments 128 to 130, wherein the one or more gene loci comprise APC, ATM, BRCA1, BRCA2, BARD1, BRIP1, CDK12, CHEK1, CHEK2, FANCA, FANCL, NF1, PALB2, PPP2R2A, PTCH, RAD51B, RAD51C, RAD51D, RAD54L, RB, TP53, VHL, or any combination thereof.
132. The non-transitory computer-readable storage medium of any one of embodiments 129 to 131, wherein a structural feature of a variant sequence comprises a substitution mutation (point mutation), an intragenic deletion, an insertion/deletion (indel) mutation, a splice site alteration, or a copy number alteration.
133. The non-transitory computer-readable storage medium of any one of embodiments 129 to 132, wherein a functional effect of a variant sequence comprises a missense mutation, a nonsense mutation, a deleterious mutation, a synonymous mutation, a non-synonymous mutation, a truncating event, a non-truncating event, a frameshift mutation, a non-frameshift mutation, an exon skipping event, or a copy number loss event.
134. The non-transitory computer-readable storage medium of any one of embodiments 129 to 133, wherein if a first variant sequence of the two or more variant sequences comprises a truncating event and a second variant sequence of the two or more variant sequences comprises a mutation that restores an open reading frame, a comparison of the structural features and functional effects of the truncating event and the open reading frame event is executed, and the reversion mutation is classified, based on the comparison, as: (i) a possible missense mutation that restores a deleterious mutation in the same codon, (ii) a possible non-frameshift deletion spanning a nucleic acid sequence region comprising a deleterious mutation in a gene locus, or (iii) a possible second frameshift indel that restores the open reading frame that was disrupted by a first frameshift indel.
135. The non-transitory computer-readable storage medium of embodiment 134, wherein if the truncating event comprises a nonsense substitution mutation, the mutation that restores the open reading frame comprises a missense substitution mutation, and a starting base position and an ending base position for the nonsense substitution mutation are located at a same corresponding amino acid position as the missense substitution mutation, the reversion mutation is classified as a missense mutation that restores a deleterious mutation in the same codon.
136. The non-transitory computer-readable storage medium of embodiment 134, wherein if the truncating event comprises a frameshift insertion mutation, the mutation that restores the open reading frame comprises a missense substitution mutation, and a starting base position and an ending base position for the missense substitution mutation are located at a same corresponding amino acid position as the frameshift insertion mutation, the reversion mutation is classified as a missense mutation that restores a deleterious mutation in the same codon.
137. The non-transitory computer-readable storage medium of embodiment 134, wherein if the truncating event comprises a frameshift deletion mutation, the mutation that restores the open reading frame comprises a missense substitution mutation, and a starting base position and an ending base position for the missense substitution mutation are located at a same corresponding amino acid position as the frameshift deletion mutation, the reversion mutation is classified as a missense mutation that restores a deleterious mutation in the same codon.
138. The non-transitory computer-readable storage medium of embodiment 134, wherein if the mutation that restores the open reading frame comprises a non-frameshift deletion mutation, and a starting base position and an ending base position for the truncating event are located between a starting base position and an ending base position of the non-frameshift deletion mutation, the reversion mutation is classified as a non-frameshift deletion spanning a nucleic acid sequence region comprising a deleterious mutation in the gene locus.
139. The non-transitory computer-readable storage medium of embodiment 134, wherein if the mutation that restores an open reading frame comprises a non-frameshift deletion mutation, and the truncating mutation comprises an insertion mutation having a starting base position and an ending base position that are between a starting base position and an ending base position for the non-frameshift deletion mutation, the reversion mutation is classified as a non-frameshift deletion spanning a nucleic acid sequence region comprising a deleterious mutation in the gene locus.
140. The non-transitory computer-readable storage medium of any one of embodiments 129 to 133, wherein if both a first variant sequence and a second variant sequence of the two or more categorized variant sequences comprise a truncating event, a comparison of the structural features or functional effects of the first truncating event and the second truncating event is executed, and the reversion mutation is classified, based on the comparison, as a possible second frameshift indel that restores an open reading frame that was disrupted by a first frameshift indel.
141. The non-transitory computer-readable storage medium of embodiment 140, wherein if both the first truncating event and the second truncating event are indel mutations, the indel mutation corresponding to the second truncating event occurs at a position upstream from a stop codon for the indel mutation corresponding to the first truncating event, and if a net change in sequence length caused by the first and second truncating events is divisible by 3, the reversion mutation is classified as a second frameshift indel that restores an open reading frame that was disrupted by a first frameshift indel.
142. The non-transitory computer-readable storage medium of any one of embodiments 129 to 133, wherein if a first variant sequence of the two or more variant sequences comprises a deleterious truncating event and a second variant sequence of the two or more variant sequences comprises a splice site alteration, a comparison of the structural features or functional effects of the deleterious truncating event and the splice site alteration is executed, and the reversion mutation is classified, based on the comparison, as a splice site alteration affecting the same exon as the deleterious truncation event.
143. The non-transitory computer-readable storage medium of any one of embodiments 129 to 133, wherein if a first variant sequence of the two or more variant sequences comprises a deleterious truncating event and a second variant sequence of the two or more variant sequences comprises an intragenic deletion, and the starting base position and ending base position of the truncating event is between the starting base position and ending base position of the intragenic deletion, a comparison of the structural features or functional effects of the deleterious truncating event and the intragenic deletion is executed, and the reversion mutation is classified, based on the comparison, as the intragenic deletion spanning a nucleic acid sequence region comprising the deleterious truncating event.
144. The non-transitory computer-readable storage medium of any one of embodiments 129 to 133, wherein if a first variant sequence of the two or more variant sequences comprises a truncating event and a second variant sequence of the two or more variant sequences comprises a copy number loss event, a comparison of the structural features or functional effects of the truncating event and the copy number loss event is executed, and the reversion mutation is classified, based on the comparison, as a possible copy number loss of a complete exon comprising the truncating event.
145. The non-transitory computer-readable storage medium of embodiment 144, wherein if the truncating event has a starting base position and an ending base position located between a starting base position and an ending base position for the copy number loss event, and the copy number loss event comprises loss of a complete exon, the reversion mutation is classified as a copy number loss of a complete exon comprising the truncating event.
146. The non-transitory computer-readable storage medium of any one of embodiments 129 to 133, wherein if a first variant sequence of the two or more variant sequences comprises a truncating event and a second variant sequence of the two or more variant sequences comprises a non-truncating event, a comparison of the structural features or functional effects of the truncating event and the non-truncating event is executed, and the reversion mutation is classified, based on the comparison, as a possible synonymous mutation occurring at the same amino acid position as a deleterious mutation.
147. The non-transitory computer-readable storage medium of embodiment 146, wherein if the truncating event comprises a synonymous or nonsense mutation and the non-truncating event comprises a missense mutation, and wherein if the truncating and non-truncating events occur within a same codon, the reversion mutation is classified as a synonymous mutation occurring at the same amino acid position as a deleterious mutation.
148. The non-transitory computer-readable storage medium of embodiment 146, wherein if the truncating event comprises a frameshift insertion mutation and the non-truncating event comprises a synonymous mutation, and wherein if the non-truncating synonymous mutation comprises an insertion that occurs within a same nucleotide range as the truncating frameshift insertion, the reversion mutation is classified as a synonymous mutation occurring at the same amino acid position as a deleterious mutation.
149. The non-transitory computer-readable storage medium of embodiment 146, wherein if the truncating event comprises a frameshift deletion mutation and the non-truncating event comprises a synonymous mutation, and wherein if the non-truncating synonymous mutation comprises an insertion that occurs within a same nucleotide range as the truncating frameshift deletion, the reversion mutation is classified as a synonymous mutation occurring at the same amino acid position as a deleterious mutation.
150. The non-transitory computer-readable storage medium of any one of embodiments 129 to 133, wherein if a first variant sequence of the two or more variant sequences comprises a truncating event and a second variant sequence of the two or more variant sequences comprises a splice site alteration, a comparison of the structural features or functional effects of the truncating event and the splice site alteration is executed, and the reversion mutation is classified, based on the comparison, as a possible intragenic deletion spanning a nucleic acid sequence region comprising a deleterious mutation.
151. The non-transitory computer-readable storage medium of embodiment 150, wherein if the splice site alteration comprises an intragenic deletion that results in in-frame exon skipping of the exon in which the truncating event occurs, the reversion mutation is classified as an intragenic deletion spanning a nucleic acid sequence region comprising a deleterious mutation.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a sample from a subject;
ligating one or more adapters onto one or more nucleic acid molecules from the plurality of nucleic acid molecules;
amplifying nucleic acid molecules from the plurality of nucleic acid molecules;
capturing nucleic acid molecules from the amplified nucleic acid molecules;
sequencing, using a sequencer, the captured nucleic acid molecules to obtain a plurality of sequence reads that represent the captured nucleic acid molecules, wherein one or more of the plurality of sequencing reads overlap one or more gene loci within a subgenomic interval in the sample;
receiving, at one or more processors, sequence read data for the plurality of sequence reads;
identifying, using the one or more processors, a gene locus of the one or more gene loci for which the gene locus comprises two or more variant sequences;
categorizing, using the one or more processors, the two or more variant sequences in the gene locus according to one or more attributes of the two or more variant sequences;
comparing, using the one or more processors, the one or more attributes of the two or more categorized variant sequences in the gene locus;
classifying, using the one or more processors, the two or more categorized variant sequences in the gene locus based on the comparison, wherein the classification indicates whether the two or more categorized variant sequences comprise a reversion mutation; and
identifying the reversion mutation in a genomic profile associated with the subject.

2-5. (canceled)

6. The method of claim 1, wherein the one or more attributes of the two or more variant sequences comprise a structural feature or functional effect.

7-9. (canceled)

10. The method of claim 1, further comprising generating, by the one or more processors, a report indicating whether or not a reversion mutation in present in a gene locus of the one or more gene loci.

11. (canceled)

12. A computer-implemented method comprising:
receiving, at one or more processors, sequence read data for a plurality of sequence reads that overlap one or more gene loci within a subgenomic interval in a sample from a subject;
identifying, using the one or more processors, a gene locus of the one or more gene loci for which the gene locus comprises two or more variant sequences;
categorizing, using the one or more processors, the two or more variant sequences in the gene locus according to one or more attributes of the two or more variant sequences;
comparing, using the one or more processors, the one or more attributes of the two or more categorized variant sequences in the gene locus; and
classifying, using the one or more processors, the two or more categorized variant sequences in the gene locus based on the comparison, wherein the classification indicates whether the two or more categorized variant sequences comprise a reversion mutation.

13. The computer-implemented method of claim 12, further comprising identifying the reversion mutation in a genomic profile associated with the subject.

14. The computer-implemented method of claim 12, wherein the one or more attributes of the two or more variant sequences comprise a structural feature or functional effect.

15. The computer-implemented method of claim 12, wherein the one or more gene loci comprise a tumor suppressor gene or fragment thereof, a homologous recombination repair gene or fragment thereof, or any combination thereof.

16-38. (canceled)

39. The computer-implemented method of claim 12, further comprising selecting a cancer treatment for the subject based on the classification of the reversion mutation.

40. The computer-implemented method of claim 39, wherein the cancer treatment comprises a treatment for a cancer caused by mutations in one or more genes of a homologous recombination repair (HRR) pathway.

41. (canceled)

42. The computer-implemented method of claim 40, wherein the cancer treatment comprises use of a poly (ADP-ribose) polymerase inhibitor (PARPi), a platinum compound, a targeted immunotherapy treatment, or any combination thereof.

43. The computer-implemented method of claim 12, wherein the detection and classification of the reversion mutation is performed without any manual curation of the sequence read data.

44-45. (canceled)

46. The computer-implemented method of claim 12, further comprising generating, by the one or more processors, a report that comprises a list of reversion mutations detected in the sample.

47-49. (canceled)

50. The computer-implemented method of claim 13, further comprising selecting an anti-cancer agent, administering an anti-cancer agent, or applying an anti-cancer treatment to the subject based on the genomic profile.

51. A method of selecting an anti-cancer treatment for a cancer in a subject, comprising:
responsive to detecting and classifying a reversion mutation in a sample from the subject, selecting an anti-cancer treatment for the subject, wherein the reversion mutation is identified and classified according to the computer-implemented method of claim 12.

52. The method of claim 51, wherein the anti-cancer treatment comprises an anti-cancer treatment for a cancer caused by mutations in one or more genes of a homologous recombination repair (HRR) pathway.

53. (canceled)

54. The method of claim 52, wherein the anti-cancer treatment comprises use of a poly (ADP-ribose) polymerase inhibitor (PARPi), a platinum compound, a targeted immunotherapy treatment, or any combination thereof.

55. A method of treating a cancer in a subject, comprising:
responsive to detecting and classifying a reversion mutation in a sample from the subject, administering an effective amount of an anti-cancer therapy to the subject, wherein the reversion mutation is identified and classified according to the computer-implemented method of claim 12.

56. The method of claim 55, wherein the anti-cancer treatment comprises a treatment for a cancer caused by mutations in one or more genes of a homologous recombination repair (HRR) pathway.

57. (canceled)

58. The method of claim 56, wherein the anti-cancer treatment comprises use of a poly (ADP-ribose) polymerase inhibitor (PARPi), a platinum compound, a targeted immunotherapy treatment, or any combination thereof.

59. A system comprising:
one or more processors; and
a memory communicatively coupled to the one or more processors and configured to store instructions that, when executed by the one or more processors, cause the system to:
receive sequence read data for a plurality of sequence reads that overlap one or more gene loci within a subgenomic interval in a sample from a subject;
identify a gene locus of the one or more gene loci for which the gene locus comprises two or more variant sequences;
categorize the two or more variant sequences in the gene locus according to one or more attributes of the two or more variant sequences;
compare the one or more attributes of the two or more categorized variant sequences in the gene locus; and
classify the two or more categorized variant sequences, based on the comparison, wherein the classification indicates whether the two or more categorized variant sequences comprise a reversion mutation.

60-65. (canceled)
